(19) United States
(12) Patent Application Publication
(10) Pub. No.: US 2024/0331820 A1
(43) Pub. Date: Oct. 3, 2024

(54) COMMUNITY BASED INDIVIDUALIZED HEALTH PLATFORMS

(71) Applicant: Prosumer Health, Hartford, CT (US)

(72) Inventors: Charles Burger, Brewer, ME (US); Don Holmes, Plano, TX (US); Gordon Jardin, Burlington (CA); Andrea Borondy Kitts, Glastonbury, CT (US); George Reigeluth, Hartford, CT (US)

(73) Assignee: Prosumer Health, Hartford, CT (US)

(21) Appl. No.: 18/624,767

(22) Filed: Apr. 2, 2024

Related U.S. Application Data
(63) Continuation-in-part of application No. 16/398,448, filed on Apr. 30, 2019, now Pat. No. 11,948,668.
(60) Provisional application No. 62/664,926, filed on Apr. 30, 2018.

(51) Int. Cl. G16H 10/60

(52) U.S. Cl. CPC G16H 10/60

(57) ABSTRACT

A system for providing an individualized health platform including a data and analytics system with patient records including a data structure having a top level problem view organized by health and care issues including current and past chronic and acute problems, a detail level problem view for each chronic and acute problem organized by each step in a diagnostic, treatment and management process, a top level status view of the health and care issues, a detail level status view of each health and care issue, and a patient's social and environmental health determinants, an adaptive knowledge engine for importing health and clinical data for analyzing the health records and assessing diagnostic, treatment, and management options, a terminal for interacting with the system, data collection devices coupled to the terminal, and a directory of vetted community and virtual-based social service organizations for addressing the social and environmental health determinants.

### RELATED APPLICATIONS

This application is a Continuation in Part of U.S. Utility application Ser. No. 16/398,448, filed on 30 Apr. 2019, which claims the benefit of U.S. Provisional Application No. 62/664,926, filed on 30 Apr. 2018, the contents of all of which are incorporated by reference in their entirety.

### FIELD

The disclosed exemplary embodiments are directed to a health management system, and in particular, to a dynamic community based, health and care management system that is customized, or individualized, specifically for each participating individual.

### BACKGROUND

Traditionally, there are difficulties in delivering high quality health and care efficiently and at a reasonable cost. The present uncoordinated, poorly integrated health and care efforts are inaccessible, complex, expensive, inefficient, and error prone.

The year after year deterioration of health outcomes across the board—e.g. overall mortality, maternal and infant mortality, rising rates of a growing list of chronic diseases, that are poorly managed, growing rates of mental disorders and a substance abuse epidemic-lead to less than desirable care, health and cost outcomes for millions each day. Improving the health of the population is supposed to be what the whole public health and healthcare endeavor is all about. Instead, there has been a steady decline of the most important measures of the health of the population compared to itself over time and to other developed countries.

For many years the mental health and substance abuse situation has been getting worse and worse. The recent outbreak of the opioid addiction epidemic, the Covid-19 epidemic, and the rising rates of teenage mental health problems are only the worst manifestations of this long festering set of problems.

Of particular note, is that of all the professions, healthcare professionals have the highest rates of mental health issues, substance abuse and suicide.

For decades the decline of the physical condition of the public health infrastructure has been well documented—e.g. the underinvestment in basic maintenance has led to a deterioration of public water systems, solid waste disposal systems and other systems.

There are many without access to basic healthcare insurance, millions more who are under-insured, who have the wrong kind of insurance and/or who must pay such high deductibles that they are unable to use the insurance they do have and thus do not see clinicians when they should for acute, chronic and/or preventive care.

For multiple reasons, there has been a growing shortage of physicians and nurses, making it difficult for those who do have insurance to get access to care, particularly at the primary care level and especially for low-income people.

Even for those who do have insurance and can get access to care, the quality and safety of that care has been declining for years leading to higher and higher rates of preventable medical mistakes, which have for some time been the third highest cause of death. In 2015, 440,000 people died of preventable medical mistakes in the hospital setting alone. This figure does not include deaths in other settings, such as primary care, other ambulatory care, home care, etc. Nor does it include the number of people who are injured, but not killed, by medical mistakes. Worst of all, there is no system for tracking, measuring and diagnosing the root causes of these errors, making it difficult to determine the size of the problem, or develop approaches for preventing these errors.

It is very difficult for an individual to have a basic understanding of costs, prices, who pays for what, why and for what reasons. As a result, it is not possible to inject market discipline and the efficiencies of typical market mechanisms into healthcare because normal dynamic market data about changing supply and demand and resulting prices do not exist.

Most reimbursements are still provided based on a volume of care provided, not on a value or quality and cost of the care delivered. And while value-based payment (VBP) systems are starting to replace fee-for-service (FFS) or volume-based systems, most providers are still reluctant to make this change because they lack the data, tools, and systems to help their patients become more involved in their care and thus improve their health outcomes, which would lead to a reduction in the use of traditional health care services and their related costs.

The ability to pay for and provide decent long-term care for the growing millions of elderly people whose life spans continue to increase year after year is a part of the health, care and cost crisis that no one wants to talk about. The result is that the overwhelming burden of long-term care falls on family members and/or poorly paid home healthcare workers many of whom have no political power or representation.

Healthcare costs are at an all-time high, for example, approximately $4.2 trillion per year or 20% of the US GNP, with unfavorable health outcomes. It is estimated that about a quarter of the costs or over $1 trillion per year are wasted on poor communication, coordination, duplicative and unnecessary care, delays, errors, corruption, administrative complexity and general inefficiencies.

An individual may be under the care of several different heath care providers, each of which may request that the individual provide a health and care history, typically by filling out a form. However, the individual may not know or remember all the details of their health and care history, and the form may not be detailed enough and most likely will not be customized to capture pertinent data that may be specific for that particular individual. Furthermore, in most cases, an individual's health and care records are not stored together and are typically scattered among the individual's various health care providers. Even when an individual's health and care records are stored together, the records are usually organized in vertical stacks by record type, such as laboratory results, images, medications, tests performed, etc., which makes it difficult to understand what different types of care an individual has received for a specific health problem and if that care is the right care.

It would be advantageous to provide a dynamic health and care management system that for each participating patient, and provide an individualized community based health and care management platform, that addresses these shortcomings and others of the healthcare environment.

### SUMMARY

The disclosed embodiments are directed to Community-based, Integrated individualized Health Platforms (CIHPs) that operate to organize, integrate, and coordinate, care and health around each patient's whole body and mind. Each human body is a system of interdependent biological organ systems. What goes on in one system effects what goes on in the other systems. To deliver optimum care and health promotion, it would be advantageous to approach each new health and care encounter in a holistic manner, considering all aspects of the health and care previously and presently being administered to every part of an individual patient in the context of the relevant peer reviewed health/clinical literature.

The disclosed embodiments allow a patient to have 24/7 access to their CIHP that, when considering an acute or chronic problem, weighs all aspects of the patient's previously experienced health and care actions and experiences when proposing guidance options for shared decision making between the patient of the CIHP and their health coach and other members of their care team, that is, anyone who may participate in the health care of the patient. A patient may utilize various health monitoring devices, for example, portable, digital home monitoring devices, to monitor many different physiological processes—e.g. blood glucose, INR, blood pressure, temperature, heart rate, weight, movement, breathing rate, etc., and the disclosed embodiments may provide the CIHP the ability to vet and then input the information from these devices into the CIHP's AI analytics engine for analysis in the context of all of the patient's data and in the context of the relevant peer reviewed literature. The data from the remote patient monitoring devices (patient terminal 105ₙ) is also entered into the patient's individualized problem-oriented health record, organized in a manner that provides a view of the patient's health as a whole and for each acute/chronic health problem in chronological order.

Machine and software-based, semi-automated health/clinical content development processes and systems with natural language processing and semantic parsing technologies may be utilized to read, parse and extract data and knowledge fragments from the voluminous numbers of peer reviewed journal articles, text books, survey reviews for keeping the CIHPs up-to-date with the most recent health and clinical research findings, data and guidance on best practices.

The disclosed embodiments may employ artificial intelligence to analyze new data on a patient's changing health in the context of all the other data in that patient's health record and in the context of the relevant health/medical literature to produce refinement questions on the initial data on a new chronic/acute problem, and thus provide individualized guidance options, and the peer reviewed references where the guidance options came from. The CIHP also provides ongoing documentation about what is going on with the patient's CIHP, recognition of patterns in the data, continuous system learning based on the flow of new data, identification of best practices for that individual and better health, care, and cost outcomes and findings.

Behavioral psychology, specifically behavioral economics, may be employed to provide insights into how decisions are made and how to shape and change individual behavior. Advances in the data, learning, decision, and cognitive sciences may contribute to promoting understanding of the nature of human cognitive limits and biases, thus causing errors in human decision making by clinicians and patients, and how all these factors interact to effect individual behavior and related care, health and cost outcomes.

New care and health models may provide new insights into how these advances in other domains can be brought to bear in health and care to help patients of the CIHPs become more engaged in their care and health—e.g. a Continuous, Collaborative Distributed Care Model that reflects the reality that care and health are continuous endeavors which often require collaborative efforts of multiple people in an ongoing manner. [Again, do we need a picture here]

It is clear that a new system and paradigm are required that work from the bottom up-one patient at a time—and whose whole purpose and mission is devoted to helping each patient optimize their health and healthcare, as opposed to the financial interests of payers and providers. Patients utilizing the disclosed embodiments may address more of their own health and care issues themselves with the help of the present system, thus becoming both providers and consumers—or the new “prosumers”—of their own health and care efforts.

The disclosed embodiments are directed to providing a patient with a dynamic, learning, community-based, integrated individualized health platform, including collecting a patient's health and care data, organizing the collected health and care data into a problem oriented health record, drafting health goals and a care plan for the patient with the aid of a health coach, and using the peer reviewed health and clinical content to vet the patient's draft goals and plans to ensure that they follow best medical practices from that content. Patients may then use their CIHPs 190 to address new acute or chronic health and care issues by responding to refinement questions coming to their smart phone CIHP app from the literature about the new issue. The patient's answers to those questions are then analyzed by an AI analytics engine in the context of all of the data in that patient's problem oriented health record and in the context of the relevant peer reviewed literature to produce near real time guidance options for shared decision making through smart telehealth calls by the patient, their health coach and other members of the patient's care team.

The method may include vetting the health goals and care plan using a data and analytics system that operates to assure that the health goals and care plan are supported by applicable peer reviewed health and clinical literature, producing individualized guidance options for achieving the health goals and adhering to the care plan based on the patient's problem oriented health record, monitoring the patient's achievements with respect to the health goals and adherence to the care plan, and providing additional guidance options based on the patient's achievements and adherence to the care plan

Collecting the patient's health and care data may include collecting data from one or more electronic medical records and patient health monitoring devices.

Collecting the patient's health and care data may include determining the patient's individual social and environmental determinants of health.

Organizing the collected health and care data into a problem oriented health record may include organizing the collected health and care data for each problem chronologically according to diagnostic, treatment and management guidance, and processes used to address particular medical issues.

Organizing the collected health and care data into a problem oriented health record may include organizing data relating to a particular health and care issue as a series of micro-care encounters for each problem arranged in chronological order.

Vetting new data coming into the patient's health platform or vetting the health goals and care plan may include using the data and analytics system to analyze peer reviewed health and clinical literature to produce health and clinical analytics and individual health and care knowledge elements, establishing standards for quality of the data inputs and the peer reviewed health and clinical literature, and vetting the health goals and care plan by comparing the health goals and care plan to the data in the peer reviewed health and clinical literature to identify possible gaps in care.

The method may further include vetting the set of determined health goals and care plan by a patient's physician and by the AI analytics engine to identify possible gaps in care.

Monitoring the patient's achievement of the health goals and the patient's adherence to the care plan may include working with the health coach to carryout health activities for achieving the patient's health goals and care plan.

Monitoring the patient's achievement of the health goals and the patient's adherence to the care plan may include inputting data that shows when a care plan activity is complete and that a health goal has been accomplished.

Providing additional guidance options may result from the AI analytics engine analyzing the data from answers to the refinement questions and screening surveys in the context of all of the data in the patient's problem oriented health record and in the context of the applicable peer reviewed health and clinical literature to produce ongoing guidance options in response to new acute and chronic issues for the patient to discuss in shared decision making through a smart telehealth call with the patient's primary care physician (PCP) and health coach and/or other members of the patient's health team as needed

The method may further include receiving additional data from the patient, and using the data and the AI analytics system to analyze the additional data and identify a potential health problem, which causes the patient to receive on their smart phone one or more of a set of refinement questions from the peer reviewed literature about a new or emerging potential acute health problem, or a screening survey for the potential chronic health problem, wherein the screening survey poses refinement and qualifying questions derived from the peer reviewed health and clinical literature applicable to the potential health problem, perform an analysis of the answers to the refinement questions in the context of the data in the patient's problem oriented health record, individual social and environmental health determinants, and in the context of the peer reviewed health and clinical literature related to the new health problem to determine further individualized guidance options for shared decision making and action.

The method may further include using the further individualized guidance options in shared decision making to plan further health and care steps for resolving the health care problem.

The method may further include documenting and storing the individualized guidance options, the health goals and care plan, data related to the patient's progress towards the health goals and the patient's adherence to the care plan, the additional guidance options resulting from the shared decision making, the further individualized guidance options, and further health and care steps in the patient's problem oriented health record. All of the data generated on a given CIHP by the patient, the health coach and other members of the patient's health team is documented and made available in real time to the members of the of the health team to identify what works and what doesn't, lessons learned which are fed back to the AI analytics engine for continuous learning and process/system improvement.

The health coach may communicate with the patient to ensure that the patient carries out the health and care activities agreed to as a result of the guidance options and the shared decision making and discussions. The health coach and the patient will use the Directory of Community/Virtual-based Social Services to help the patient carryout what has been agreed upon that needs to be done to address each new acute and chronic health issues and to help the patient overcome their social determinants of health, such as lack of transportation, poor access to care, fresh food, exercise facilities, socialization and company, etc.

The method may further include providing the patient and other members of the patient's health team (with the patient's prior consent) with access to their problem oriented health record at all times.

In other aspects the disclosed embodiments are directed to a system for providing a patient with an individualized health platform, including a data and analytics system having one or more patient problem-oriented health records including a data structure with a top level problem view organized by health and care issues including a patient's current and past chronic and acute problems, a detail level problem view for each chronic and acute problem organized by each step in a diagnostic, treatment and management process, a top level status view of the health and care issues, a detail level status view of each health and care issue, and a patient's social and environmental health determinants, an adaptive knowledge engine configured to automatically import health and clinical data for use in analyzing the one or more patient problem-oriented health records and assessing diagnostic, treatment, and management options for the health and care issues, a patient terminal for providing the patient with an ability to interact with the system, one or more patient data collection devices coupled to the patient terminal, and a directory of community and virtual-based social services comprising vetted social service organizations for use by the patient in addressing one or more of the patient's social and environmental health determinants.

In further aspects of the disclosed embodiments, a method of providing a patient with an individualized health platform includes receiving agreement to terms and conditions for use from the patient, assigning a health coach to the patient, receiving permission to access the patient's medical practitioner records from the patient, collecting the patient's previously undocumented health and care data, collecting the patient's individual social and environmental health determinants, using a data and analytics system to organize the collected health and care data into a problem oriented health record for the patient comprising records relating to a particular health and care issue arranged as a series of micro care encounters comprising units of measure or analysis relating to the particular health and care issue and organized in order of each step in a diagnostic and treatment and management process in chronological order, and using an adaptive knowledge engine to automatically import health and clinical data for use in analyzing the problem-oriented health record and assessing diagnostic, treatment, and management options for the health and care issues.

The following sections describe the unique components of the system providing the community-based individualized health platforms, how each of the components works and how the overall system works, how a hypothetical patient may use the system to address different care and health issues, and the benefits of the system.

### BRIEF DESCRIPTION OF THE DRAWINGS

FIG. 1 shows a schematic illustration of an exemplary health management system providing community based individualized health platforms according to the disclosed embodiments;

FIG. 2 shows a block diagram of an exemplary data collection device utilized by the disclosed embodiments;

FIG. 3 shows a block diagram of an exemplary on-boarding process for providing a community based individualized health platform to a patient, according to the disclosed embodiments;

FIG. 4 shows an exemplary illustration of a patient's individual social and environmental health determinants according to the disclosed embodiments;

FIG. 5 shows a block diagram of a continuation of the exemplary on-boarding process of FIG. 3;

FIG. 6A illustrates examples of data types suitable for inclusion in a patient's dynamic integrated personal health record;

FIG. 6B shows an exemplary display of a dynamic integrated personal health record on a mobile device;

FIG. 6C illustrates a data structure of a patient's dynamic integrated personal health record;

FIG. 7 illustrates a top level problem view of a data structure of an exemplary dynamic integrated personal problem oriented health record;

FIG. 8 illustrates a detail level problem view of a particular health and care issue in the data structure;

FIG. 9 illustrates a top level status view of health and care issues, where the health and care issues are organized according to their present various active and inactive status indications;

FIG. 10 illustrates a detail level status view of a particular health and care issue;

FIG. 11 illustrates an alternate detail level status view of a particular health and care issue;

FIGS. 12 and 13 illustrate a block diagram of an adaptive knowledge engine according to the disclosed embodiments;

FIG. 14 shows an example of a directory of community and virtual based social services;

FIGS. 15-21 illustrate examples of system communications using mobile devices;

FIGS. 22-24 illustrate examples of system communications using a patient terminal;

FIG. 25 illustrates an exemplary overview of how, once health goals and care plan are set, a patient may proceed to carry out daily health activities;

FIG. 26 shows how a data and analytics system may operate to identify a particular health problem from new data entered by a patient;

FIG. 27 illustrates that each patient may connect external information and databases to their dynamic integrated personal health record;

FIG. 28 illustrates a summary of the set up steps, various assets, and particular health and care issues that may be managed for a specific patient;

FIG. 29 illustrates a summary of how the system operates on an ongoing basis to address changes to the particular health care issues; and

FIG. 30 illustrates at least some of the advantages provided by the disclosed health and care management system

### DETAILED DESCRIPTION

The aspects and advantages of the exemplary embodiments will become apparent from the following detailed description considered in conjunction with the accompanying drawings. It is to be understood, however, that the drawings are designed solely for purposes of illustration and not as a definition of the limits of the invention, for which reference should be made to the appended claims. Additional aspects and advantages of the invention will be set forth in the description that follows, and in part will be obvious from the description, or may be learned by practice of the invention. Moreover, the aspects and advantages of the invention may be realized and obtained by means of the instrumentalities and combinations particularly pointed out in the appended claims.

### System Overview

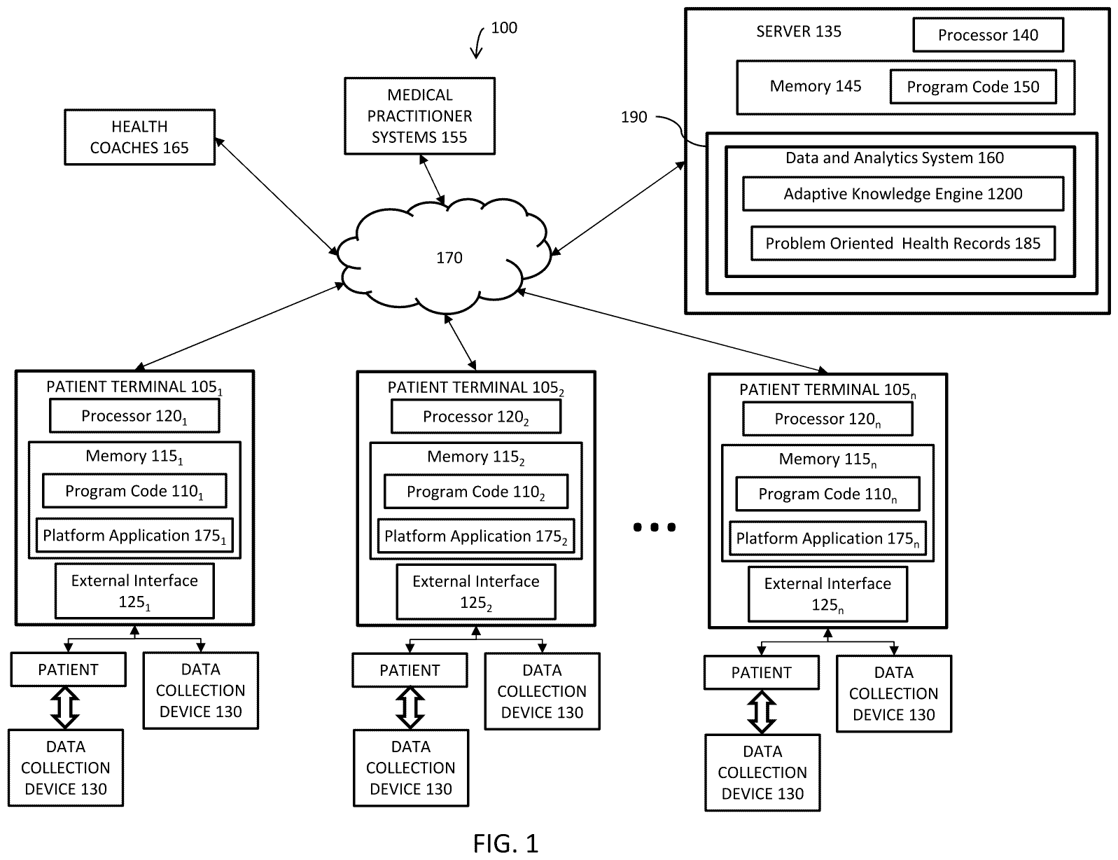

FIG. 1 shows a schematic illustration of an exemplary health and care management system 100 that implements Dynamic, Community Based, Individualized Health Platforms (CIHPs) 190 according to the disclosed embodiments.

The health and care management system 100 may include one or more servers 135, each with a processor 140, and memory 145 storing computer program code 150 for generally operating the health and care management system 100. The one or more servers 135 may be managed by cloud computing services with suitable encryption and data privacy/security features and may include any commercially available cloud services. Part of the data privacy and security capabilities result from separating all of a patient's personal identification data from their medical, care and health data.

For purposes of the disclosed embodiments, the server 135 may implement the CIHPs 190 which may include a data and analytics system 160 and patients' dynamic integrated personal problem oriented health records, also referred to as Problem Oriented Health Records (POHRs) 185. The data and analytics system 160 may further include an adaptive knowledge engine 1200 that operates to organize in an integrated fashion, and link, vetted health and care knowledge fragments, health and clinical analytics, input from an assigned health coach 165, input from the patient, and content in the POHRs 185, to provide ongoing, real time evidence-based individual healthcare guidance options.

Each patient's POHR 185 may be stored on the server 135 and may be managed by a cloud computing provider with suitable encryption and data security features. The overall architecture of the CIHPs 190 may provide further security for the patient's data by storing the patient's personal identifier data on the patient's patient terminal 105 and storing the patient's health and clinical data on the cloud-based server with a coded reference key linking the two sets of data. Each patient's POHR 185 may be implemented as an integrated health and care data base that may include all an individual's providers, health insurance details, medical history and problems, including problem list, dental and eye care and mental health, vital signs, history, nutrition, exercise, allergies, medications, blood type, etc. integrated in one record, securely accessible by multiple heath care professionals across the care continuum—e.g. wellness, prevention, early detection, chronic disease management and acute care episodes. By having all an individual's health and care data in one POHR 185, the health and care management system 100 may ensure that every time a patient has a new care/health issue the CIHP 190 may assess that issue in the context of all that person's data—not just part of it as is the case today—and in the context of the relevant health/medical literature. Each piece of data may be time and date stamped and coded in such a way that all the data that relates to a particular subject, such as diabetes type 2, can be assembled and analyzed together.

### Health Coaches

Each patient may have a designated health coach 165 that may interact with a patient when they want to communicate with a person about a health-related issue, for example, a chronic disease management issue, an emerging/existing acute problem, an insurance issue or an administrative issue. The health and care management system 100 health coaches 165 may receive training and may have certain qualifications and certifications. For example, the health coaches may have taken one of the recognized and approved Health Coaching accreditation and certification courses, successfully completed that course and received their certificate of completion. The health management system coaches 165 may operate to gain the confidence and trust of the patients they work with through motivational approaches, empathy and compassion, and by following through on, and closing out each of the health and care tasks the health coaches 165 commit to perform for the patients, and closing out each of the tasks the patient commits to carrying out. If successful, a patient will be able and want to become more involved in achieving their health goals and care plan in the manner that works best for them. To build up this confidence and trust may take time and lots of little steps and nudges, and each patient will have different triggers for involvement, different strengths and weaknesses, different preferences and psychologies, families, chronic diseases and family and work situations. The health and care management system 100 gives the patient and their health coach many opportunities to build this trust through the enrollment and planning processes that they perform together. In these many integrated and interdependent ways, the CIHPs 190 give the patients the tools and the agency that they need to be able to take more personal responsibility for their own health and care and thus find their own path to improved health and care.

### Medical Practitioner Systems

The medical practitioner systems 155 may generally include one or more processors and memory storing computer program code for generally operating secure online portals for managing patient records, and enabling patients to have personalized access to their patient records, communicate with medical practitioners, and schedule, reschedule, and cancel appointments. The medical practitioner systems 155 may be managed by cloud computing services with suitable encryption and data privacy/security features and may include any commercially available cloud services.

### Patient Terminals

The health and care management system 100 may include one or more patient terminals 105₁-105ₙ. Each patient can access their own CIHP 190 at anytime from anywhere through patient terminals 105₁-105ₙ—e.g. for example, a desktop computer, a laptop, a tablet, a mobile phone, or any other computing device capable of performing the functions of the disclosed embodiments. It should be understood that a patient may utilize more than one patient terminal 105₁-105ₙ, and more than one type of patient terminal 105₁-105ₙ, to access the patient's CIHP 190. For example, a patient may use a mobile phone at one point in time to access the system and later may use a tablet for system access. It should also be understood that a patient terminal, with the proper permissions, may be shared by a patient, their health coach, a medical practitioner, and/or other members of their care team, or other patients, whom the patient gives permission to access their CHIP, and may be located at, for example, a patient's residence, a medical practitioner's office, a hospital, or any suitable location with internet access.

Each patient terminal 105₁-105ₙ may include computer readable program code 110₁-110ₙ stored on at least one non-transitory computer readable medium for carrying out and executing the processes described herein. In at least one embodiment, the computer readable program code 110₁-110ₙ may implement a platform application 175₁-175ₙ for providing, in combination with other components of the community-based, dynamic, learning, individualized health and care management system 100, access to the CIHPs 190. The computer readable medium may be memories 115₁-115ₙ, and in alternate aspects, the computer readable program code 110₁-110ₙ may be stored in memories external to, or remote from, patient terminals 105₁-105ₙ. Memories 115₁-115ₙ may include magnetic media, semiconductor media, optical or voice/audio media, or any media which is readable and executable by a computer. Each patient terminal 105₁-105ₙ may also include a processor 120₁-120ₙ for executing the computer readable program code 110₁-110ₙ.

Each patient terminal 105₁-105ₙ may also include at least one external interface 125₁-125ₙ. In at least one embodiment, the at least one external interface 125₁-125ₙ may include a keyboard, mouse, touch screen, display camera, microphone, voice recognition system, or any device or combination of devices suitable for providing a patient with an ability to interact with the community-based, dynamic, learning, individualized health and care management system 100. In some embodiments, the at least one external interface 125₁-125ₙ may include a wireless interface, for example, Wi-Fi 802.11, Bluetooth 802.15, cellular 2G-5G, or any other suitable wireless interface. Some embodiments may also include a wired interface, for example, Ethernet, Universal Serial Bus (USB), Serial Advanced Technology Attachment (SATA) or any other suitable wired interface. The external interface may also provide a patient with the ability to input health and care data for example, patient perceived symptoms and data from data collection device 130, which may include for example, one or more remote medical monitoring devices, such as portable, digital home monitoring devices for monitoring various physiological processes—e.g. blood glucose, international normalized ratio (INR) blood tests, blood pressure, temperature, heart rate, breathing rate, etc., environmental sensors, or any devices suitable for use as part of the community-based, dynamic, learning, individualized health and care management system 100. In some embodiments, the external interface 125 may directly connect to the one or more data collection devices 130 and may automatically collect the information from these devices, including an individual's favorite nutrition and/or fitness apps. In some embodiments, the external interface 125 may include a web based user interface.

### Data Collection Devices

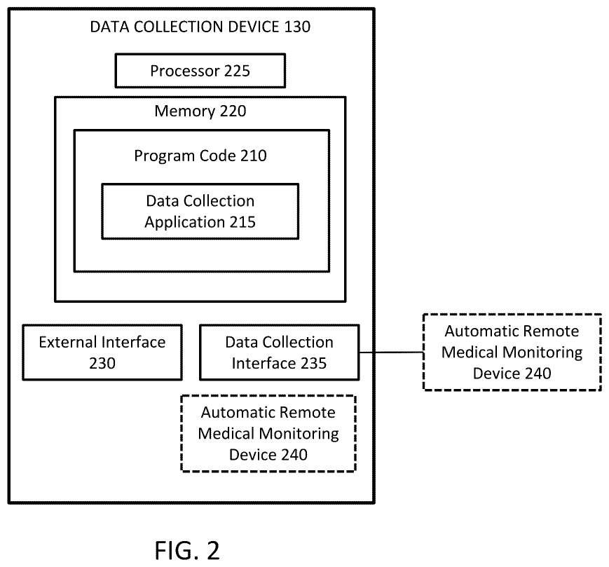

FIG. 2 shows a block diagram of a data collection device 130 according to the disclosed embodiments. Each data collection device 130 may include computer readable program code 210 stored on at least one non-transitory computer readable medium for carrying out and executing the processes described herein. In at least one embodiment, the computer readable program code 210 may implement a data collection application 215 for collecting data from a patient. The computer readable medium may be a memory 220 and in alternate aspects, the computer readable program code 210 may be stored in a memory external to, or remote from, the data collection device 130. Memory 220 may include magnetic media, semiconductor media, optical or voice/audio media, or any media which is readable and executable by a computer. Each data collection device 130 may also include a processor 225 for executing the computer readable program code 210.

Each data collection device 130 may also include at least one external interface 230. In at least one embodiment, the at least one external interface 230 may include a keyboard, mouse, touch screen, display camera, microphone, voice recognition system, or any device or combination of devices suitable for providing a patient with an ability to interact with the community-based, dynamic, learning, individualized health and care management system 100. In some embodiments, the at least one external interface 230 may include a wireless interface, for example, Wi-Fi 802.11, Bluetooth 802.15, cellular 2G-5G, or any other suitable wireless interface. Some embodiments may also include a wired interface, for example, Ethernet, Universal Serial Bus (USB), Serial Advanced Technology Attachment (SATA) or any other suitable wired interface.

The external interface 230 may also provide a patient with the ability to input health and care data for example, patient perceived symptoms and data from standalone devices, such as blood pressure monitors, pulse oximeters, thermometers, glucometers, activity trackers, weight measurement devices, heart rate monitors, electrocardiogram (ECG) monitors, or any other standalone devices suitable for patient monitoring.

Each data collection device 130 may also include at least one data collection interface 235 that may provide an interface to one or more automatic remote medical monitoring devices 240. The automatic remote medical monitoring devices 240 may include medical monitoring devices that automatically monitor and provide data to the data collection interface 235 about physiological aspects of a patient, for example, blood pressure, blood oxygen levels, patient temperature, environment temperature, blood glucose levels, patient activity, weight, heart rate, electrocardiogram data, sleep patterns or any other measurable physiological aspects of a patient.

Data collected by the external interface 130 and the data collection interface 235 may be provided to the data collection application 215 which may operate to condition, format, or standardize the patient's physiological data for submission to an Artificial Intelligence (AI) analytics engine for analysis in the context of all of the patient's data and in the context of the relevant peer reviewed literature. The data collected by the external interface 130 and the data collection interface 235 may also be entered into a patient's individualized personal problem-oriented health record, organized in a manner that provides a view of the patient's health as a whole and by problem.

Utilization of automatic remote medical monitoring devices advantageously reduces manual data entry errors, late or nonexistent transmission of measures, and ensures that the patient's physiological data will be processed so that current and emerging health problems may be identified and addressed more efficaciously.

A patient may generally understand how to use each data collection device 130, may consult a user's manual or other literature, or may be trained by the health coach to use each data collection device 130. Each data collection device may be configured to automatically measure and send collected data continuously or periodically, such that the patient does not have manual record results, which may increase convenience and accuracy. Each data collection device 130 may optionally be paired with a digital tablet which may communicate with the system 100.

### Medication Management System

The health and care management system 100 may also include a medication management system that includes a particular data collection device 130 in the form of a medication dispenser. The medication management system provides comprehensive, efficient, robust, efficacious support to help patients improve their medication adherence. Specifically, after an onboarding process, described below, the patient may receive a medication dispenser with different wells for different daily time periods, for example, morning, noon, afternoon, and night, into which patients may put their medications. In the bottom of each well, there is a computer chip that is activated each time the door on the top of each well is opened. When a patient opens the door for the morning pills, the computer chip sends a signal to the system 100 and the adaptive knowledge engine 1200, that those medications have been dispensed. If the patient is late opening up the well door, the chip will send out a beep to remind the patient to take their pills. If the patient does not take their pills after the beep, then the chip will produce five additional beeps until the patient takes the medications. If the patient does not respond to any of the beeps, then the health coach will send a text message to the patient. If no response, then the health coach will call the patient, and if still no response, the health coach will send someone to the patient's house to make sure that everything is okay. This escalation system for helping patients to manage their medications addresses one of the many problems with current health care delivery by helping patients improve their adherence to the medication management part of their care plan, which improves the management of the acute and/or chronic health issues that the patient is dealing with and in turn results in better care, health and cost outcomes.

The various components of the health and care management system 100, including the patient terminals 105₁-105ₙ, one or more servers 135, medical practitioner systems 155, and one or more health coaches 165 may communicate over a network 170. In some aspects, the data collection devices 130 may communicate directly with the one or more servers 135 through the network 170.

### On Boarding Process

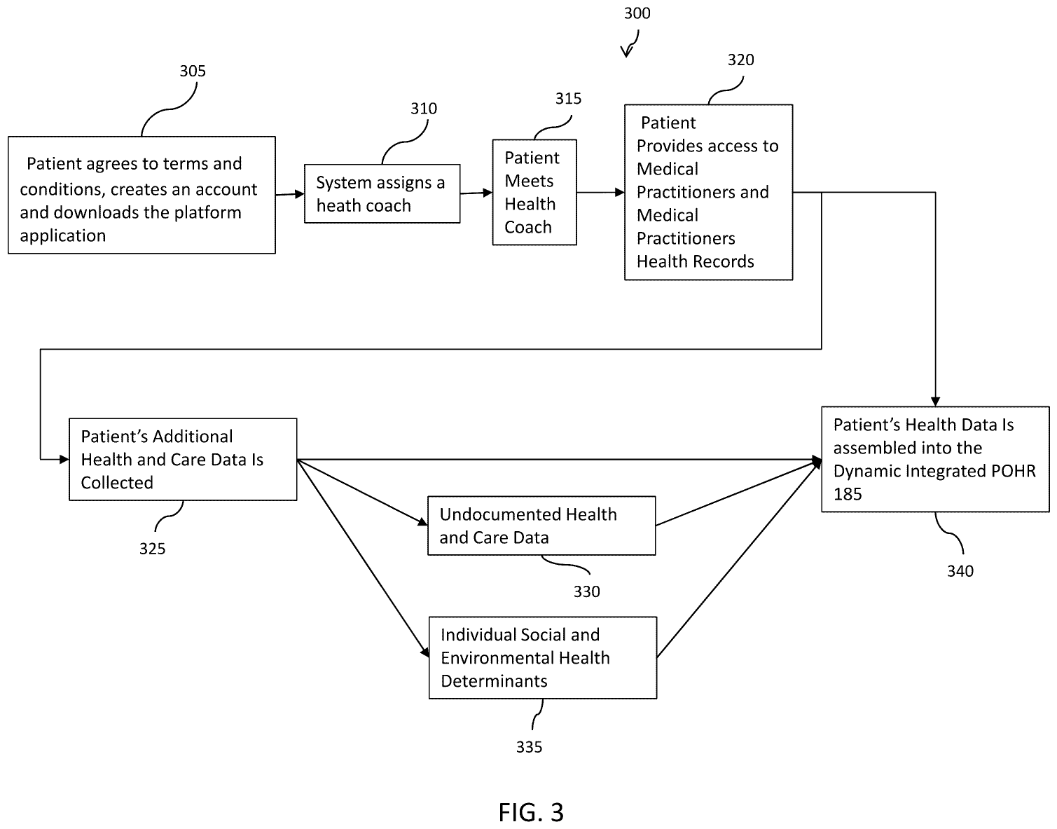

FIG. 3 shows a block diagram of an exemplary on-boarding process 300 for providing a CIHP 190 for a patient.

The platform application 175 may provide patients with the ability to enroll and to provide enrolled patients with access to their CIHPs 190. A typical onboarding process 300 for enrolling a patient into the health and care management system 100 is shown in FIG. 3.

An important first step in establishing health goals and care plans is to for the patient and the health coach to begin to get to know each other—e.g. an open discussion about where the patient is with their life, their challenges, difficulties, positive aspects, their job, etc. After establishing some rapport and initial trust, then the patient and the health coach may be able to begin to discuss health goals and care plans, if the patient is ready to do that. Health goals and care plans are determined by having the patient and their health coach discuss the data in the patient's POHR 185, once established, and the health circumstances, priorities and constraints of the patient—e.g. is the patient married, have children, mentally and/or physically disabled, or suffer from substance abuse, or unemployed, without a steady income, does the patient have a strong social network living nearby, does the patient live in a dangerous neighborhood without access to parks and/or public transportation, live in a house with mold or other ambient air quality problems, broken windows, poor security, etc. Data on these social determinants of each person's health (SDOH, discussed below) are collected and define what the patient's situation is. It is key in establishing health goals to meet the patient where they are and to be realistic about the issues they have to deal with on a day to day basis. A Directory of Community/Virtual-based Social Services, also discussed below, may be able to address some of these issues, such as inadequate access to transportation or fresh food or exercise facilities, while others will be more difficult to address in the short term—e.g. poor housing, dangerous neighborhood, etc. If the patient is ready, the patient and the health coach can have an initial discussion about which of these SDOH issues are the most important and how to go about beginning to address the top two or three issues.

The on boarding process may also layout what current acute and/or chronic health issues the patient is dealing with—e.g., obesity, hypertension and/or diabetes type 2, and/or a recently sprained wrist. From this data, it is possible to begin to discuss what the patient's priorities are with respect to this set of health issues. To some extent the relative degree of acuity or seriousness of the issues may dictate these priorities somewhat—e.g., if the patient's blood glucose is badly out of control or their systolic blood pressure is consistently around the 200 level, those issues will need to take higher priority than some other less serious/dangerous health issue. But even with serious issues it is important to not try to do too much too quickly, which can lead to failure to achieve goals and disappointment. So, the health coach tries to work with the patient to establish a small number of realistic goals, that are obtainable within a relatively short period of time.

Once these initial health goals are agreed upon, the health coach and patient begin to put together a care plan for each of the goals starting with data about what the patient is currently doing—e.g. taking certain medications, going to the gym once a week, etc. This data on the patient's current care plan will be read and vetted by the AI analytics engine to see if there are any gaps in care—e.g., taking the wrong medication, etc. Based on this data, again it is important that the patient address high priority care plan issues first and not try to do too much too soon—e.g. if the patient has been experiencing high blood pressure for several weeks and has been taking their medication, then one of the care plan issues is to talk with the patient's PCP about maybe changing medications as the AI analytics engine has recommended. There may be other issues to discuss with the PCP as well, but this persistently high blood pressure issue is serious enough that it should be dealt with right away by having the health coach set up a smart telehealth call with the patient's PCP, sending the data from the patient terminal 105 blood pressure device on the persistently high blood pressure to the patient's PCP and the patient, and then having the smart telehealth call with the PCP to discuss what should be done to address the high blood pressure issue. Having dealt with this problem the patient and their health coach can then move on to establish other care plan items. The full new care plan will be vetted by first the AI analytic engine to identify serious issues and gaps in care and by the patient's PCP, who may or may not suggest changes in the plan.

The patient and their health coach then discuss and agree on a plan for implementing the plan—e.g. how will they work together each week, how will they communicate, etc. It is the health coach's responsibility to make sure that the patient stays on the plan with lots of little nudges if necessary and to discuss with the patient any issues that may arise—e.g. the patient not sticking to the nutrition part of the care plan or not taking their medication when they should, etc. The health coach will also use the Directory of Community/Virtual-based Social Services to help the patient address the SDOH issues that the patient may have, which will be identified from the SDoHs collected.

As shown in block 305, a patient may decide to use the health and care management system 100, agree to the terms and conditions governing the use the health and care management system 100, and download the platform application 175. Referring to block 310, the system may assign a health coach, and the health coach and patient may meet 315. The meeting may be an in person meeting or a virtual meeting using any virtual meeting tool.

As shown in block 320, the patient may then provide the health coach, appropriate medical practitioners, other users, and other members of their care team, with access to their health and care data, as stored on the health and care management system 100. For example, the patient may fill out HIPAA forms or may provide other forms of permission. The health and care data as stored on the health and care management system 100 may include health and care data stored on the medical practitioner systems 155, health and care data stored in POHRs 185, health and care data stored on the patient's patient terminal 105, and health and care data stored on any of the patient's collection devices 130.

Optionally, initially, the patient may express an interest in using the health and care management system 100, the health and care management system 100 may query the patient to determine health coach characteristics suitable for the patient, and may assign a health coach. The health coach and patient may meet and the health coach may then assist the patient with the on-boarding process, for example, explaining the terms and conditions, assisting with creating an account and providing required information, downloading the platform application and providing instruction as to how to operate the platform application. In some instances, the health coach may assume a role as a surrogate and operate the platform application on behalf of the patient. It should be understood that interaction between the patient and the health coach may be in person or though electronic devices, for example, mobile phone, tablet, computer, or other devices capable of providing interaction between the patient and the health coach.

As shown in block 325, additional patient health and care data is collected, for example, newly discovered or newly perceived health conditions, health and care data from previous medical practitioners, or any other known health and care data. In addition, heath and care data 330 undocumented in the health and care management system 100, such as data related to the patient's diet, mental assessment data, data related to undocumented medical conditions, data related to medications, supplements or other items being ingested by the patient, and individual social and environmental health determinants 335. As shown in block 340, the patient's medical practitioner's health records 320, the additional health and care data 325, the undocumented health and care data 330, and the individual social and environmental health determinants 335 are assembled and stored in the patient's POHR 185.

In some embodiments, the patient may provide the additional patient health and care data by answering questionnaires and queries on paper or using an automated wired or wireless device, for example, a tablet, computer, laptop, mobile phone, or any other device suitable for collecting data. In other embodiments, the health coach may assist the patient with providing the additional patient health and care data, or the health coach may assume a surrogate role and provide the additional patient health and care data on behalf of the patient.

It should be understood that not all of a patient's health and care data may be collected during the initial on-boarding process, but may be continuously collected by the health and care management system 100 over time and iteratively stored in the patient's POHR 185. In particular, health and care data collected by the health coach 165, the medical practitioner systems 155, the data collection devices 130, and health and care data provided by the patient may be continuously collected and stored in the POHR 185. The patient and the health coach may also endeavor to collect data from the patient's EMR or EMRs, which can be done by connecting the CIHP 190 to the patient's EMR/EMRs. This step is often not easy to carryout but is getting easier to do with improvements in technology and data transfer programs/APIs, etc.

### Individual Social and Environmental Health Determinants (SDOHs)

The individual social and environmental health determinants, also referred to as Social Determinants of Health (SDoHs) 335, additionally collected as part of the previously undocumented additional patient health and care data, may be stored in the patient's POHR 185.

Recent research has shown that about 80% of each person's health status is determined by their social determinants of health—e.g. their access to transportation, fresh food, fitness facilities, good housing, neighborhood, education, strong social network, etc.—and that only about 20% of a person's health status is determined by what occurs within clinics and physicians' offices. To improve individuals' health outcomes, it is, therefore, important to give them the ability to address the SDOH that are creating barriers to their achieving and maintaining good care and health. The first step in addressing a person's SDOH is to know what SDOH they have, which the CIHPs 190 do by collecting data on their SDOH when they are onboarded into their CIHP system. Each person's SDOH is coded and date and time stamped so that when a patient has an acute or chronic issue, the adaptive knowledge engine 1200 will bring the patient's data on those SDOH that the peer reviewed literature says are relevant to that issue into its assessment and preparation of guidance options for the patient, the health coach and other members of that patient's health team to use as part of their activities to address the acute or chronic issue.

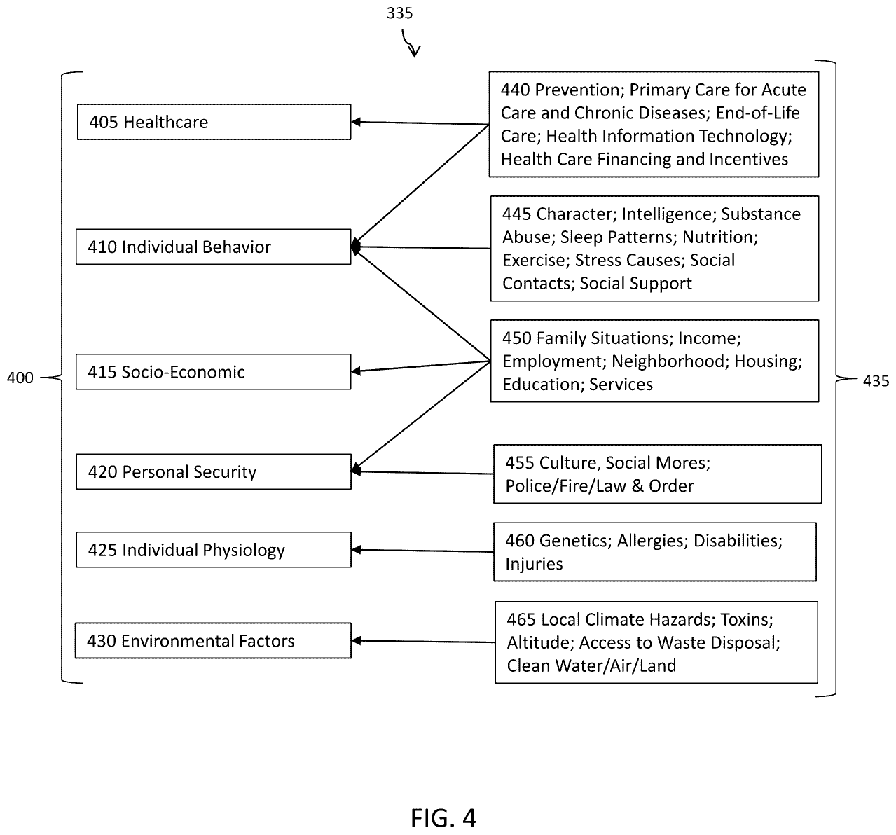

An exemplary illustration of individual SDOHs 335 that may be collected implemented in a patient's CIHP 190 are shown in FIG. 4. The SDOHs 335 may include and be organized into a main set of factors 400, for example, healthcare 405, individual behavior 410, socio-economic factors 415, personal security issues 420, individual physiology 425, and environmental factors 430. Each of the main set of factors may have a number of driving characteristics 435.

The driving characteristics for healthcare 405 may include, for example, Prevention; Primary Care Acute Care/Chronic Diseases; End-of-Life Care; Health Information Techs; Health Care Financing/Incentives 440.

Exemplary driving characteristics for individual behavior 410 may include Prevention; Primary Care Acute Care/Chronic Diseases; End-of-Life Care; Health Information Techs; Health Care Financing/Incentives 440, Character; Intelligence; Substance Abuse; Sleep Patterns; Nutrition; Exercise; Stress Causes; Social Contacts; Social Support 445, and Family Situation; Income/Employment; Neighborhood/Housing; Education/Services 450.

In particular, sleep patterns and stress are identified as important drivers of a person's health status. Peer reviewed literature has shown that lack of sleep or too much stress can be one of the many causes of many different chronic conditions, which if left untreated can undermine a person's health. When a new patient of their CIHP starts using their system, the health coach will help the patient enter data on different aspects of their health, including in particular, data on the patient's sleep patterns and stress situations.

Data collected related to sleep patterns may include measures such as the Pittsburgh Quality Sleep Index (PSQI). The PSQI is a series of questions about sleep-related behaviors and is used in both clinical and research settings. Patients may provide self-rated responses to questions, including sleep quality, sleeping disturbances, and daytime dysfunction. A medical practitioner may evaluate the responses to the questions and identify the next steps in improving the patient's quality of sleep. Rapid eye movement (REM) sleep may be measured with a data collection device 130 designed to measure sleep quantity and quality, for example, by tracking sleep stages throughout the night. More serious sleep problems may require seeing a sleep specialist and attending a clinic for specialized sleep studies and tests, which the patient's health coach can arrange.

Data related to stress may generally correspond to the different types of stress a patient may be experiencing, and the health issues associated with the different types of stress. Different types of stress may include chronic stress, life events, traumatic life events, daily stressful issues, and acute stress. Measuring stress may include a length of time someone is exposed to a stressor, the period of life and/or circumstances when experiencing the stressor, confounding/multiplicative environmental factors including SDoHs, for example, a high crime neighborhood, food insecurity, isolation, exposure, dysfunctional/abusive family situation.

Stress data may be collected during the onboarding process when identifying an individual's social determinants of health and may include data on the type of health response each stressor causes—e.g. psychological, behavioral, cognitive, physiological, and the strength of the effect, its duration and manifestation. Measures may also be obtained from commercially available systems, for example, The Stress Measurement Network Toolbox that provides validated and curated stress measures (https://stresscenter.ucsf.edu/).

Upon the identification of sleep or stress issues, the health coach may help identify health goals and care plans related to the sleep or stress issues, the system may provide facilities where the patient may indicate that they have carried out the agreed upon activities to address their sleep and/or stress issues, and the data collection devices 130 may measure how well the patient's sleep and/or stress issues are being dealt with.

Driving characteristics for socio-economic factors 415 may include, for example, Family Situation; Income/Employment; Neighborhood/Housing; Education/Services 450.

Driving characteristics for personal security issues 420 may include, for example, Family Situation; Income/Employment; Neighborhood/Housing; Education/Services 450, Culture, Social Mores; Police/Fire/Law & Order 455.

Driving characteristics for individual physiology 425 may include, for example, Genetics; Allergies; Disabilities; Injuries 460.

Driving characteristics for environmental factors 430 may include Local Climate Hazards; Toxins; Altitude; Access to Waste Disposal; Clean Water/Air/Land 465.

It should be understood that individual SDOHs 335, the main factors 405, 410, 415, 420, 425, and 430, and the driving characteristics 440, 445, 450, 455, 460, and 465 may be dynamic, and that the system 100 has the ability to discover the exemplary presently listed main factors and driving characteristics and to also incorporate additional individual SDOHs, main factors, and driving characteristics as they become known, for example, as they are identified during the on-boarding process 300 or as they are continuously collected by the health and care management system 100 over time. For example, individual SDoHs, including main factors and driving characteristics may be initially collected and may also be recognized on a continuing basis and added to a patient's POHR 185 by the health coach 165, a medical practitioner, the medical practitioner systems 155, the data collection devices 130, and the patient itself.

The health and care management system 100 thus operates to include a wide range of possible environmental and social determinants of an individual's health. Recent studies have shown that healthcare provided in clinics presently may only account for about 10% of an individual's health status, even though it may account for about 90% of the total cost of an individual's health expenditures. It should be noted that over time, the system may determine and collect additional individual social and environmental health determinants, main factors, and driving characteristics, and may determine which individual social and environmental health determinants, main factors, and driving characteristics may have more impact on a particular patient.

For example, while population level protocols and guidelines change frequently for health indicators such as cholesterol, blood pressure, lipid levels, etc., often the important thing for the individual is what level of a particular health indicator works best for them in terms of their own health situation, health and care goals and plans—e.g. it may be that for some people a blood pressure goal of 140—instead of the 120 population guideline—is more appropriate. These insights also help to drive more robust engagement by the individual in their care and health. While there is much talk about standardizing care, this is inimical to the reality that each of us has a different body with different health and care needs. Thus, the system may operate to standardize the quality of inputs so that the system can individualize, rather than standardize, care using a combination of the patient's health and care history, and the patient's individual social and environmental health determinants and the relevant health/medical literature.

### Continued On Boarding

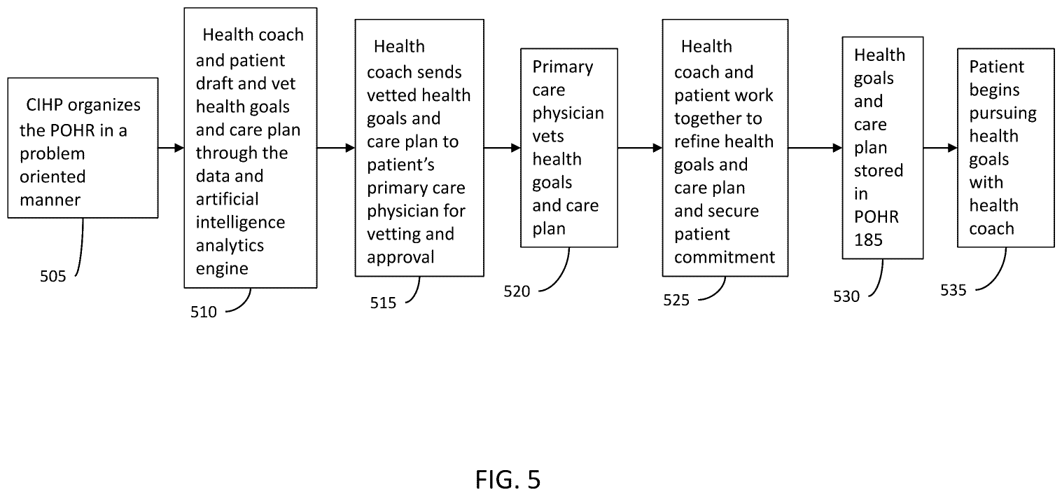

FIG. 5 shows a block diagram of a continuation of the exemplary on-boarding process of FIG. 3.

Once the patient's data has been assembled into the POHR 185, and as additional data is added over time, the CIHP 190 may organize the POHR 185 in a problem oriented manner as shown in block 505. The organization of the POHR 185 improves the operations of the overall system, and in particular the operations of the data and analytics system 160 because the organization improves data retrieval times and reduces the time required to assemble the patient's data into views illustrating relationships among different health and care entries. For example, the POHR 185 may be organized by health and care issues, and a particular health and care issue record may include a record of symptoms, a collection of tests, analyses of the tests, treatment procedures, medications prescribed during those procedures, surgery records, rehabilitation programs and other records, organized together to provide a comprehensive medical history of the patient, organized according to a particular health and care issue experienced by the patient over time, resulting in less computing time and resources required by the processor 140 and memory 145, and less time required to analyze the POHR 185 information. In some embodiments, the records may be further organized in a time line, for example, in order of each step in the diagnostic and treatment and management process. As another example, all the data relating to a particular health and care issue may be displayed as a series of micro-care encounters and may show each item or event that occurred during each micro-care encounter. These micro-care encounters may then be arranged chronologically to determine how the particular health and care issue has been addressed over time, at least reducing system resources required to assemble data pertinent to the particular health and care issue.

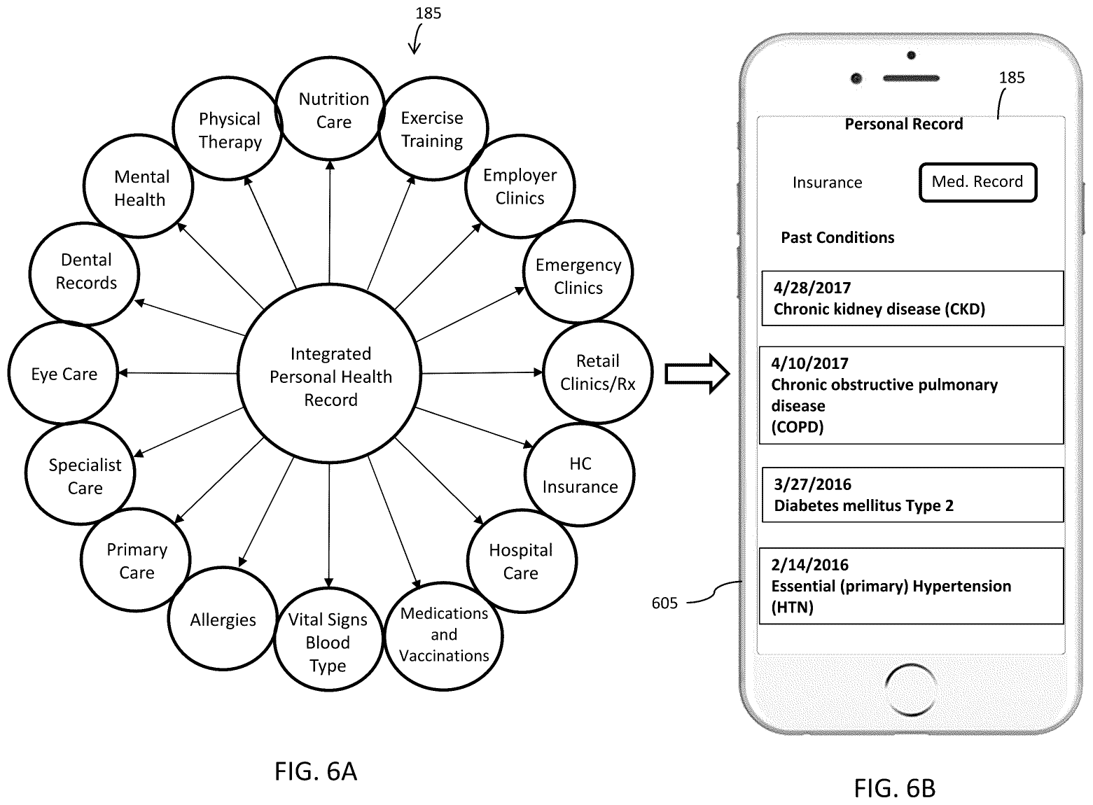

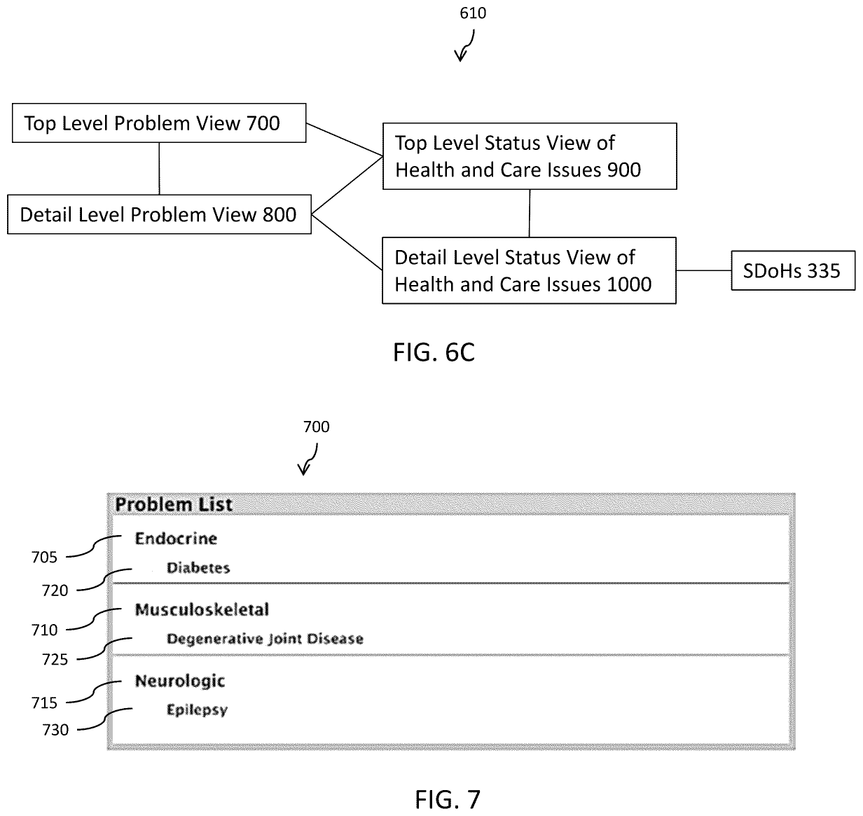

FIG. 6A illustrates examples of data types, including medical records, types of care, SDoHs, main factors, driving characteristics, health facilities, and any other data types suitable for inclusion in a patient's POHR 185. FIG. 6B shows an exemplary display of a POHR 185 on a mobile device 605, and FIG. 6C illustrates a data structure 610 of a patient's POHR 185 that may include a top level problem view 700, a detail level problem view 800, a top level status view of health and care issues 900, a detail level status view of health and care issues 1000, and that patient's SDoHs.

FIG. 7 illustrates the top level problem view 700 of the data structure 610 of an exemplary POHR 185, organized by health and care issues, in particular at a top level, organized by bodily systems, in this example, endocrine 705, musculoskeletal 710, and neurological 715 systems. A particular patient's problems with each bodily system may be listed for that particular bodily system, for example, diabetes 720 may be a problem with this patient's endocrine system 705, degenerative joint disease 725 may be a problem with this patient's musculoskeletal system 710, and epilepsy 730 may be a problem with this patient's neurological system 715.

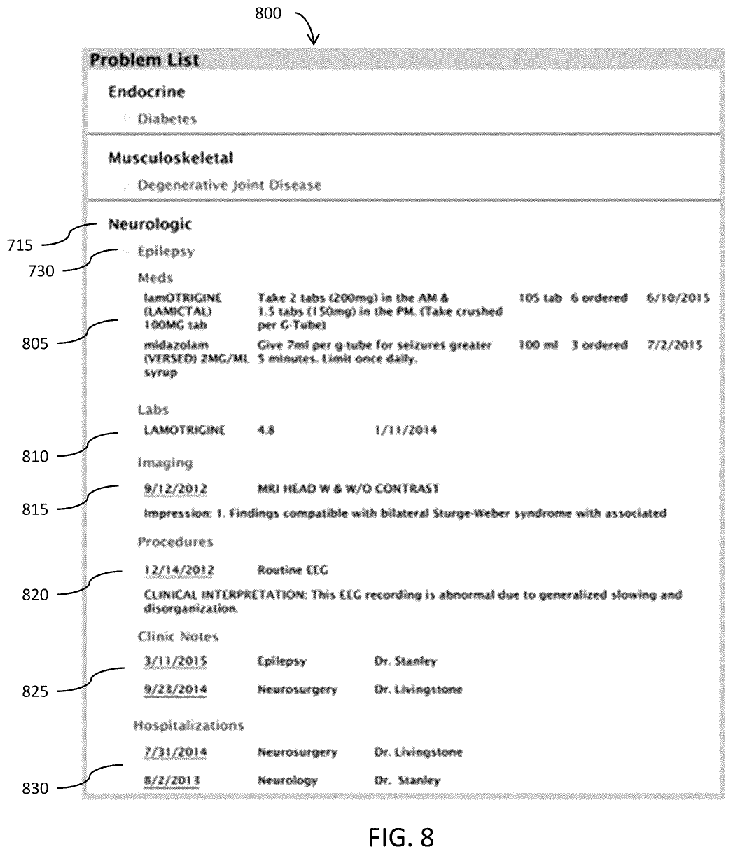

FIG. 8 illustrates the detail level problem view 800 of the epilepsy problem 730 with the neurological system 715. The detail level problem view 800 includes current medications 805, laboratory test results 810, imaging test results 815, procedures 820, clinical notes 825, and hospitalizations 830, organized in order of each step in the diagnostic and treatment and management process, as a series of micro-care encounters showing each item or event that occurred during each micro-care encounter, where the micro-care encounters may then be arranged chronologically to illustrate the steps used to address the particular health and care issue over time.

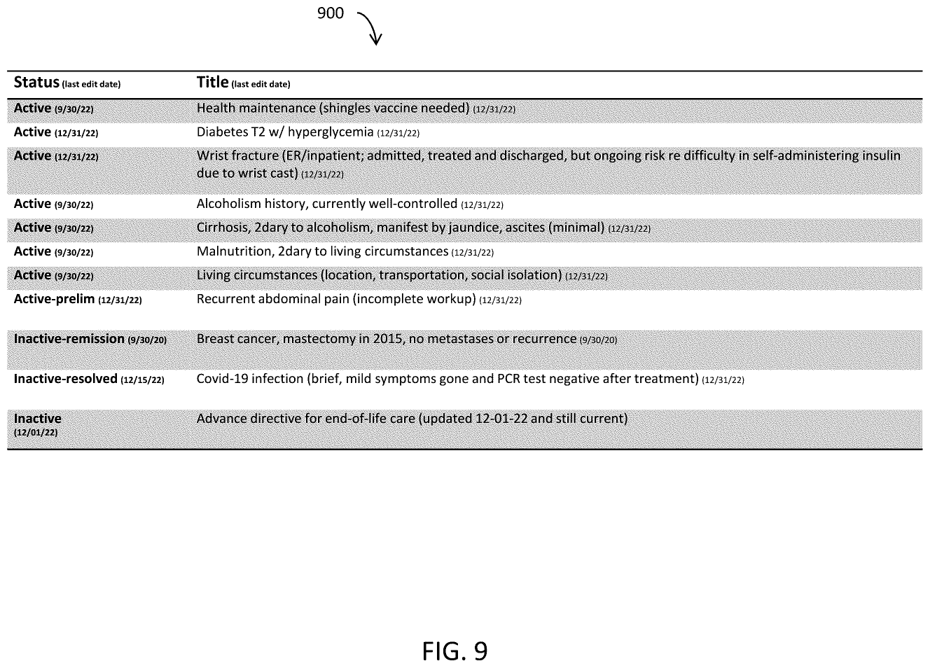

FIG. 9 illustrates the top level status view of health and care issues 900, where the health and care issues are organized according to their present various active and inactive status indications.

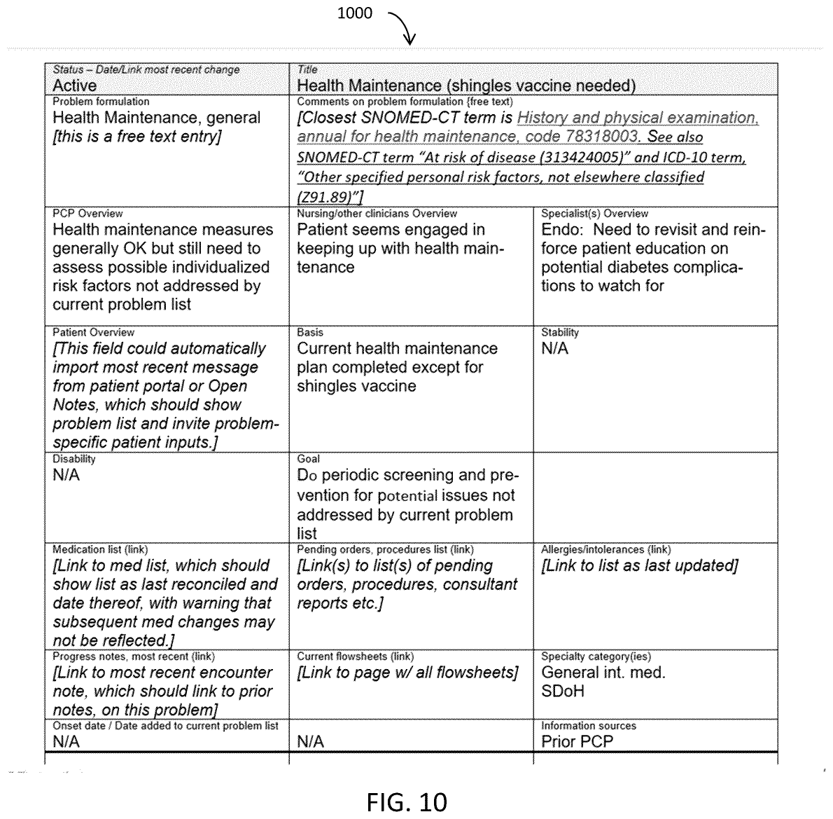

FIG. 10 illustrates a detail level status view of a particular health and care issue with various health care providers overviews, a patient overview, and links to goals, orders, medications, and other data in the detail level problem view 800, pertinent to the status of a particular health and care issue.

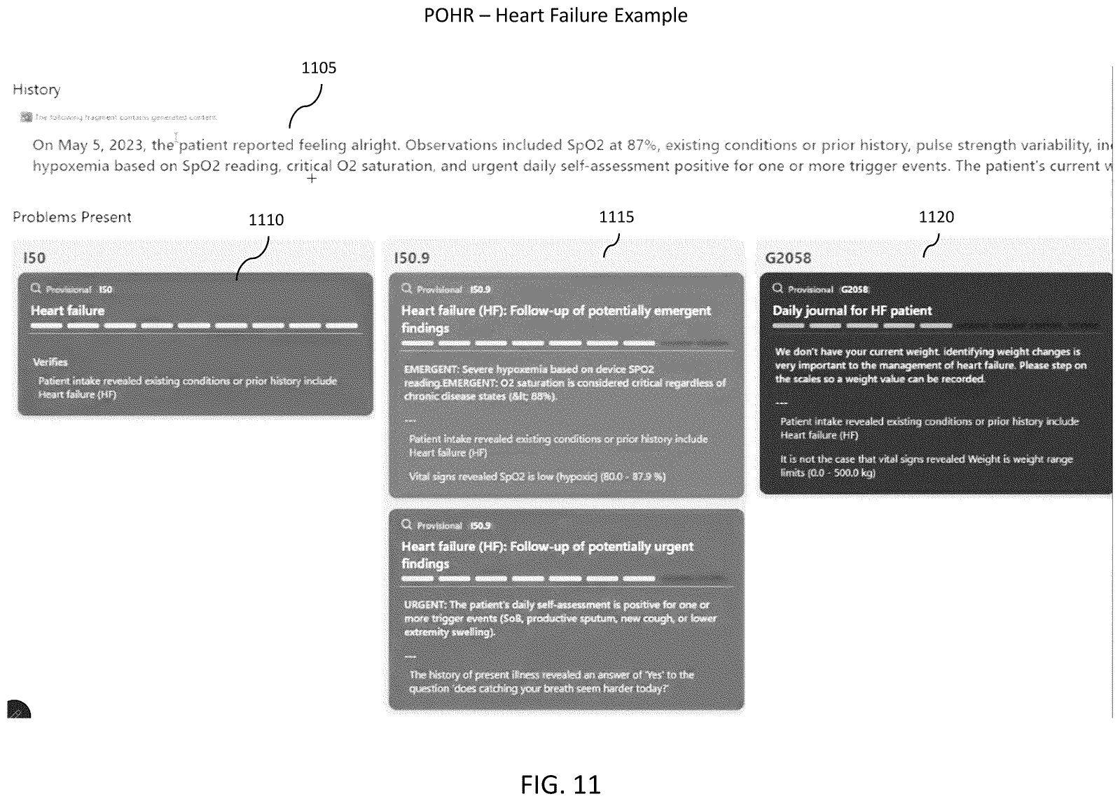

FIG. 11 illustrates an alternate detail level status view of a particular health and care issue, in this example, heart failure, with a history overview 1105, verification of the problem 1110, various findings 1115, and a patient journal 1120.

Returning to FIG. 5, the health coach and patient may draft health goals and a care plan to address the health and care issues, and vet the plan through the data and analytics system 160, as shown in block 510. Using the techniques and operations discussed below, the data and analytics system 160 may operate to vet the patient's health and care plan by comparing the patient's health and care plan against peer-reviewed health and medical literature recommended actions.

Many of the patient's health goals may be based on the patient's health and care data—e.g. if the patient has high blood pressure of 180/90, one of the patient's health goals will be to reduce their blood pressure to let's say 140/90 within three months of working together. This health goal may differ from the population guideline of 120/90 because this individual may have other health traits that make a different blood pressure goal desirable and possible. In turn, much of a patient's care plan may derive from their health goals—e.g. if one of the patient's health goals is to reduce blood pressure from 180 to 140, and this patient is an African-American female, then the care plan may stipulate that she take a calcium channel blocker to reduce her blood pressure, and not the usual recommendation of a beta blocker and/or an ACE inhibitor, because they don't work as well for African-Americans as calcium channel blockers. Once the patient and the health coach 165 have put together the draft health goals and care plan, the data and analytics system 160 and the patient's PCP may check them for accuracy, internal integrity, contra-indications and completeness. The patient and the health coach 165 may then commit to carryout and achieve the health goals and the care plan by using the patient's CIHP 190 on a daily basis if necessary and in a way that works best for the particular patient.

Health goals may be determined through a combination of the health data for a given patient—e.g. if they have high blood pressure, they will have as one of their health goals to reduce their blood pressure—and personal preferences—e.g. person is not obese or overweight, but wants to lose ten pounds, etc. Health goals may be prioritized by severity/acuity indexes developed by the data and analytics system 160 when analyzing the medical data 205, —e.g. blood pressure of 200/120 with a headache may be a malignant headache emergency and may have a higher priority than the person's blood glucose reading of 150, which is a health goal but may not be triaged as quickly as the malignant headache.

It should be understood that each entry provided by the patient, the health coach 165, the patient's PCP, a medical practitioner, the medical practitioner systems 155, the data collection devices 130, and the data and analytics system 160, related to the patient's health and care data may be collected, recorded, organized, and stored as part of the patient's POHR 185.

### Data and Analytics System

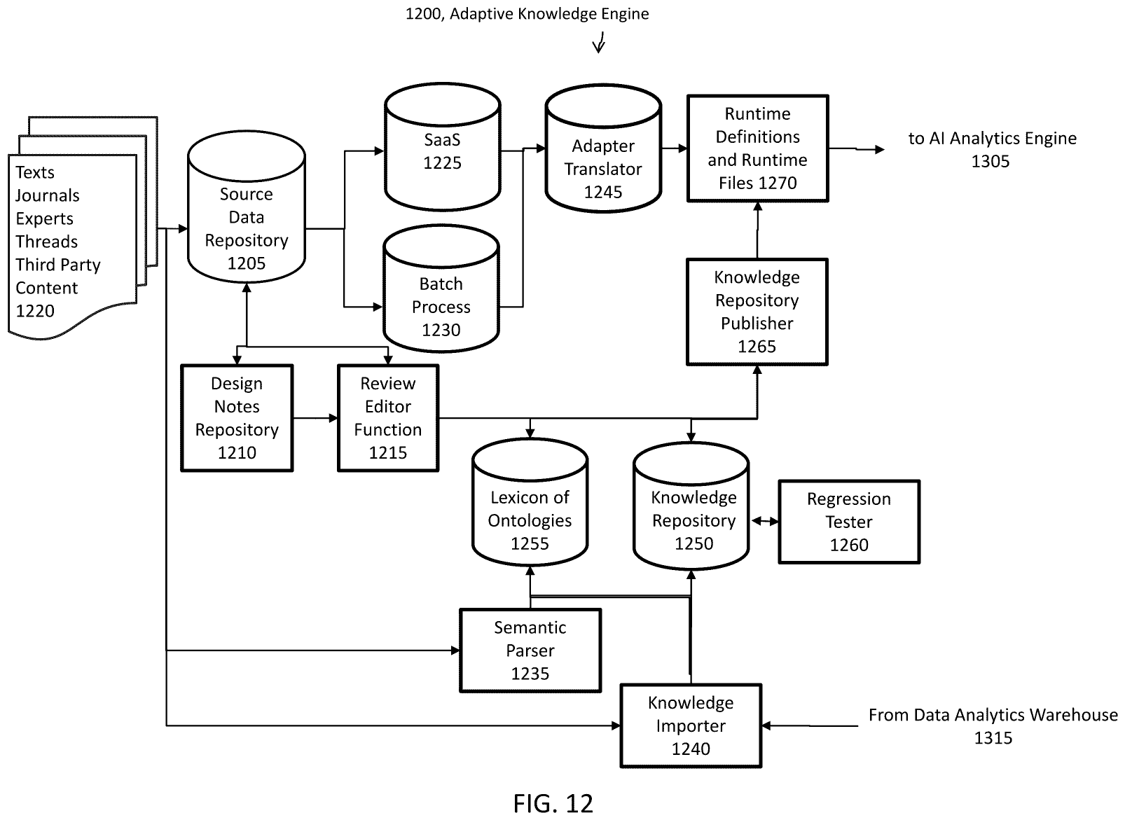

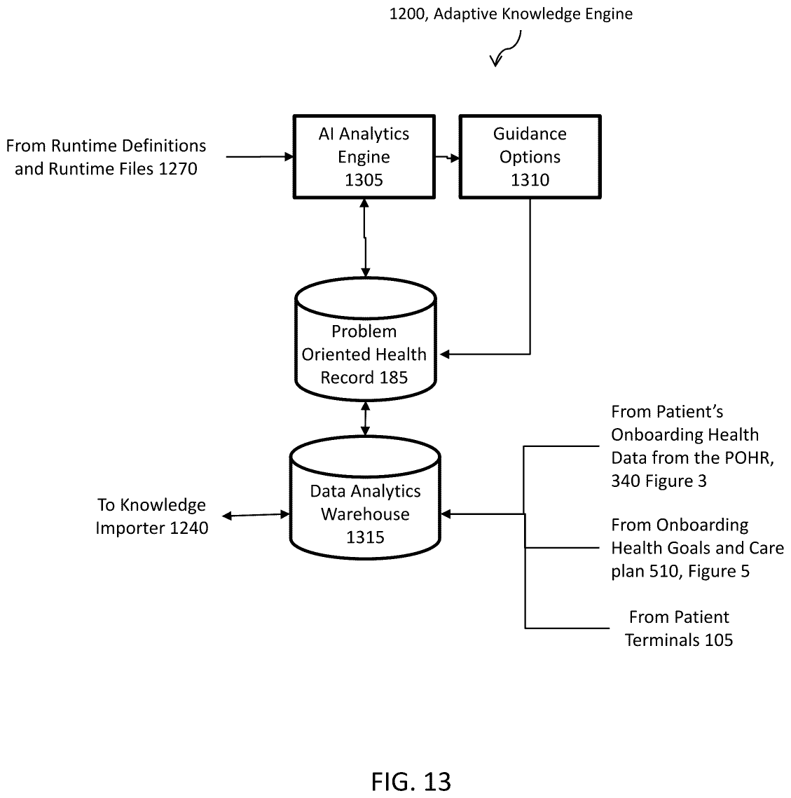

The data and analytics system 160 may include the POHR's 185 and an adaptive knowledge engine 1200, as shown in FIGS. 12 and 13.

### Adaptive Knowledge Engine

The adaptive knowledge engine 1200 operates to organize in an integrated fashion, and link, vetted health and care knowledge fragments, health and clinical analytics, input from an assigned health coach 165, input from the patient, and content in the POHRs 185, to provide ongoing, real time evidence-based individual healthcare guidance options.

The adaptive knowledge engine 1200 generally provides:
Normalized knowledge concepts in a domain so that ideally a concept need only be described in one way, with one combination of data elements;
Efficient and accurate acquisition of health and medical knowledge content;
Similarity matching between language based external knowledge and language generated from a lexicon of ontologies 1255, so that matching of concepts is accurate and efficient;
Data storage through the lexicon of ontologies 1255 in a machine language, including language models that allow concepts and content to be rendered in natural language for interpretation and review;
The ability to gather multiple external data sets into one coherent data dictionary as well as support and integrate customer creation of new vocabularies as needed to support new domains;
Support for a wide range of relationships between normalized and de-normalized data elements (string synonym, weak synonym, strong antonym, weak antonym, etc.);
Manage and ensure continuity of data element changes including effects on decision engine rules or patterns and subsequent versions of included ontologies; and;
Support easy visualization of data relationships and uses.

The adaptive knowledge engine 1200 may include various facilities for automatically importing large volumes of health and clinical data 1220 for use in analyzing a patient's integrated health and care information and assessing diagnostic, treatment and management options.

The components of the adaptive knowledge engine 1200 may be implemented in hardware, software, or a combination of hardware and software. The adaptive knowledge engine 1200 may include may include a source document repository 1205, a review editor 1215, and a design notes repository 1210. The source document repository 1205 may provide a storage memory for the health and clinical data 1220, for example, peer reviewed texts, journals, input and threads from experts, third party content, health and medical materials, articles and textbooks.

The design notes repository 1210 may operate to store note of questions about accuracy, authenticity and the provenance of new digitized peer reviewed health and medical materials, and questions about what the new materials means when the new materials are introduced into the Source Document Repository.

The review editor 1215 operates to resolve these questions or states that they cannot be resolved and that there is a certain degree of uncertainty in these findings that users of the CIHP need to be aware of. These uncertainties are stored in a knowledge repository 1250 and reported when they come up in references for different guidance options.

The adaptive knowledge engine 1200 may further include a software as a service importing system 1225, a batch processing importing system 1230, a semantic parser 1235, a knowledge importer 1240, or any other system appropriate for importing particular types of data, an adapter translator 1245, the knowledge repository 1250, and a lexicon of ontologies 1255. In some embodiments, the software as a service system 1225 and the batch processing system 1230 may generally operate on data related to individual patients, while the semantic parser 1235 and knowledge importer 1240 may generally operate on data related to populations.

The software as a service system 1225 may be used to extract bulk data from medical monitoring devices, third party applications, web sites and other sources that may be conditioned using a software as a service application. The batch processing system 1230 may typically process bulk data available in batch formats, such as EHR (Electronic Health Record), CCDA (Consolidated Clinical Document Architecture), HL7 (Health Level-7), or any other format suitable for batch processing.

The bulk data may then be vetted, curated, coded into a standard format, disaggregated, and associated with the particular integrated health record of an individual, by the adapter translator 1245. The adapter translator 1245 may also perform validity checks on the data, such as range limits for variables.

The semantic parser 1235 may operate to parse vetted published works to produce representations in predicate logic or other formal language. The formal language may be stored in the knowledge repository 1250 which may be configured to operate an automated and industrialized process for consuming health and medical knowledge and turning it into usable and accessible content. The semantic parser may further parse the vetted published works for vocabulary for use in the lexicon of ontologies 1255. The lexicon of ontologies 1255 may be configured as a descriptive ontology; hyper-structured observational data structure, used to create the clinical context of elemental data points, which is/contains an observation and its complete context.

The knowledge importer 1240 may operate to analyze structured data sets, for example, data in Unified Medical Language System (UMLS), Systemized Nomenclature Of Medicine (SNOMED), or any other suitable format for use in the knowledge repository 1250 and the lexicon of ontologies 1255. The semantic parser 1235 and the knowledge importer 1240 may also operate to vet, curate, code into a standard format, and provide validity checks on the imported data.

The adaptive knowledge engine 1200 may further include a knowledge repository publisher 1265 that may operate to publish the contents of the knowledge repository 1250 and the lexicon of ontologies 1255.

The contents of the knowledge repository 1250 and the lexicon of ontologies 1255 may be published by the knowledge repository publisher 1265 as runtime definitions and knowledge files 1270.

Turning to FIG. 13, The adaptive knowledge engine 1200 may also include an artificial intelligence analytics engine (AIAE) 1305 that may have machine learning and natural language processing functions. The AIAE 1305 may operate on the bulk data vetted and curated by the adapter translator 1245 and the contents of the lexicon of ontologies 1255 published by the knowledge repository publisher 1265 as runtime definitions and runtime files 1270, and may operate on the POHRs 185 for each patient, to generate and update the POHRs 185 as disclosed herein. The AIAE 1305 generally provides:
Pattern matching, including set intersection vs. non-intersection, identifying pattern match-based trigger patterns, nested patterns of rising complexity and patterns where combinations of elements provide weight greater than the sum of their individual parts, and pattern matches based on predictive patterns and data points;
A dual-sum calculation that reconciles positive and negative data into a ranking;
Set theory/relational algebra;
Use of a formal ontology or lexicon;
Neural classification, using layered heuristics that are evidence or science based;
Text mining;
Similarity matching;
Relational data modeling and normalization;
Space efficient, lossless data representation; and
Lossless data assessment.

The adaptive knowledge engine 1200 may operate to review the large volumes of current peer reviewed health and clinical literature in the different health and clinical areas, as stored in the lexicon of ontologies 1255 and the knowledge repository 1250, to produce health and clinical analytics and individual health and care knowledge elements that may be time stamped and coded. The health and clinical analytics and individual health and care knowledge elements may be analyzed by the AIAE 1305 to provide updated guidance options 1310 in response to data that may be introduced into a patient's POHR 185 from electronic medical records, medical devices, sensors, etc. The studies may be vetted for quality, peer review, and timeliness by a panel of experts before being processed by the adaptive knowledge engine 1200. The processed data may be coded in various ways, including according to a particular topic, such that related data, for example, data related to obesity, may be collected and the new data may be integrated with the individual's data to determine what is the health and care meaning of that new data in the context of all of the other data for that individual, and may be used in an analysis of issues which a patient's CIHP 190 may be currently addressing. As a result of the range limits for each data element, the vetting, coding, and integrating processes, the health and care management system establishes de-facto standards for the quality of the data and information processing used in analyzing the data. As a further result, each piece of guidance produced by a patient's CIHP 190 may be evidenced/best practice based, and patients may be provided with a facility to view the reasoning and references behind each piece of guidance.

As a result, transparency may be maintained throughout each CIHP 190 so that the reasoning and references from which each guidance was produced may be examined, and it may be demonstrated that the guidance options are based upon best practice evidence. The data and analytics system 160 may update the data periodically on an automatic basis by checking for new studies and literature and may also be programmed to update the data immediately if a new study comes out on a particular topic that has been identified as having particular significance for changing best practices for health and care.

The data and analytics system 160 may provide ongoing, real time evidence-based individual acute care and diagnostic, investigation, treatment and chronic disease management guidance—e.g. “What should I do next” and why, based on a combination of constantly updated medical literature sources and data in a patient's POHR 185. A patient's CIHP 190 may assess each new piece of data about a new issue in the context of all the other data in that patient's integrated POHR 185 and in the context of the relevant medical evidence that may be developed by the data and AI analytics.

One of the key limitations of AI systems is that they use algorithms that are invisible to the patients of AI systems. As a result, it is difficult if not impossible for patients to understand what the output of AI systems is based on. In other words, the inputs are not transparent to the patient and their providers and so it is not possible to know how much confidence to put in the findings of AI systems. The AIAE 1305 overcomes this lack of transparency by providing the patient with a complete explanation of why and how the guidance options that the AI system produces are created—e.g. what data is used, what analytical techniques were employed, what peer reviewed studies are the guidance options based on, etc. In addition, the AIAE 1305 tells patients such as patients, health coaches and clinicians how much confidence to place in the findings and the peer reviewed literature that supports those findings.

The approach that the present embodiments utilize with respect to AI technology in the CIHP system is able to enhance the positive aspects while reducing the negative aspects. For example, the AIAE 1305 is transparent and multi-modal as discussed above. In some embodiments, the AIAE 1305 may utilize a large language model, such as CHATGPT to translate and make intelligible the guidance options that are produced. The large language technology may be contained and ring-fenced by only allowing interaction with previously vetted or peer reviewed databases on specific health and medical topics—e.g. diseases, labs, tests, images, treatments, social determinants of health, etc. Furthermore, the present embodiments are directed to training the large language model while constraining the training set to a narrow database to only material that is fully curated and vetted.

An exemplary sequence for creating the guidance options may begin with a patient clicking on an acute issue that is bothering them—e.g. sore throat, chest pain, headache, etc.—and then the AIAE 1305 responds with a list of refinement questions that come from the peer reviewed literature, and for a chronic disease problem like low blood sugar reading, the AIAE 1305 may then send a screening survey of questions again from the peer reviewed literature about low blood sugar for diabetics. The answers to these questions are returned to the AIAE 1305 which analyzes the clinical and health meaning of the answers in the context of the rest of the patient's data in their POHR 185 and in the context of the relevant peer reviewed literature to produce guidance options about possible next steps—e.g. additional labs, tests, images, new treatments, referrals, etc.—which are used in a follow on smart telehealth call with the patient, the patient's health coach, and their primary care doctor to discuss the guidance options, what to do next, who is going to do what by when, what are the barriers to carrying out these next steps and how to remove the barriers.

The adaptive knowledge engine 1200 uses the data produced by the knowledge repository publisher 1265 that has comprehensive, up-to-date data on diseases, their precursors and causes, their different types, their complications, their labs, tests, images, treatments, and guidelines for measuring whether the disease is present. So, when a vital sign or other health measure comes into the data and analytics system 160, the adaptive knowledge engine 1200 reads the measure and compares it to what the literature says is the range within which that measure should fall. If it is outside the range, the adaptive knowledge engine 1200 will send a message to the patient that they need to redo the measure and resend it and will send the range that that measure should be within.

After a number of these observations for a given measure, the adaptive knowledge engine 1200 learns that for a given individual they can have a slightly higher or lower measure as their normal level for that measure. In this way, the guidelines become individualized and therefore more accurate, and there will be fewer false alerts because the adaptive knowledge engine 1300 will not send an alert if a measure is outside of the population guidelines but is within an individualized set of guidelines.

Currently, the overwhelming majority of guidelines for different diseases are population based not individual based. But health issues happen one individual at a time. So, using population-based guidelines represents a blunt and often misleading tool for a problem that is individual in nature. By using individual-based guidelines, chronic disease screening questions and acute issue refinement questions and individual-based data throughout each micro-care encounter and the entire CIHP produces individualized and thus more precise and accurate data going into the guidance options that the adaptive knowledge engine 1200 produces, which in turn produces higher quality decisions on the part of the CIHP, the patient, their health coach, PCP and other members of their health team. Better decisions lead to fewer preventable medical errors, fewer malpractice suits and better care, health and cost outcomes.

The screening surveys and the questions contained in them for detecting, preventing and managing/monitoring chronic diseases come from the peer reviewed literature, the resulting Knowledge Repository and again the experience of each individual patient of the CIHPs. When a patient has a measure on one of their vital signs that is outside of their individualized guidelines, like too low a blood glucose reading that can happen to diabetics, the adaptive knowledge engine 1200 recognizes that the measure is outside of the guidelines, has been that way for three straight days and is headed in the wrong direction. The adaptive knowledge engine 1200 then sends to the patient a screening survey for detecting people that are at risk of having hypoglycemia which is a serious condition that occurs with diabetics whose blood sugar level goes too low for a prolonged period.

It is not unusual for there to be doubts in medicine and health about which approach makes the most sense. The data and analytics system 160 makes these doubts transparent and obvious for all to see, while at the same time making it clear to patients, health coaches, clinicians, and other members of the patient's health team that the guidance options are based on the best practice medicine and healthcare that is currently available.

In particular, the CIHP 190 will tell the patient when the adaptive knowledge engine 1200 does not have knowledge on a particular question, disease, treatment, etc. In this way, patients may know the boundaries of the knowledge in the adaptive knowledge engine 1200.

Returning again to FIG. 5, as shown in block 515, the health coach may then send the vetted health goals and care plan to the patient's PCP for review and approval. As shown in block 520, the patient's PCP may vet and approve the health goals and care plan. The health coach 165 and the patient may work together to refine the patient's health goals and have the patient commit to the patient's health goals and care plan, and as shown in block 525, The health goals and care plan may be stored in the patient's POHR 185, as shown in block 530, and the health coach 165 and patient may begin implementing the agreed upon health and care plan, as shown in block 535.

### Directory of Community and Virtual Based Social Services for Addressing Individual SDoHs

The disclosed embodiments are further directed to providing a directory of community and virtual based social services for addressing a patient's SDoHs. This directory is a significant aspect of the services provided by the system 100 because it addresses a previously unaddressed need. Generally in the delivery of care, 50% of the time patients are not able to carry out next steps coming from individual health goals and care plans care guidance options resulting from interaction with the system 100. As a result, patients are not able to adhere to the guidance options and agreed upon next steps, which may result in their problems becoming worse, which may in turn result in disruption to their lives and the lives of their caregivers, and in some instances, require admission or readmission to a hospital with associated rising healthcare costs. To address a patient's SDoH, the patient and their health team need to know what resources are available to the patient to address those SDoH. To address this need, the disclosed embodiments include the creation of directories of vetted community/virtual-based social services that provide a catalogue of vetted organizations that may provide services for addressing each SDoH.

The first step in assembling the list of social services for a given SDoH is to do an internet search and a literature search to learn about different virtual social services for that SDoH. The next step is to do the same thing for the community-based social services for a given SDoH. Putting these two lists together provides a comprehensive starting point for the list of community/virtual social services for a given SDoH and locale. The directory is dynamic, and we add to the initial list when we hear/read about relevant social service entity for a given SDoH.

The first steps in vetting the list are to read the website of a given social service entity, read what articles we can find on that entity, evaluations of past users of the service. We then call up a given social service entity and ask to speak to a manager of the entity to have a discussion with that person about whether they would be willing to receive requests from CIHP users to use their services in particular ways. Once securing that agreement, the contract information is loaded into the Directory so that the user can click on a particular social service entity and see the name of the contact person, their telephone number, email address and street address. Then when an CIHP user uses the Directory, we ask them to evaluate the quality of the service they receive based on a questionnaire designed for that purpose. We are then able to give that entity a grade which can change based on additional evaluations by other CIHP users.

Finally, to individualize the use of the directory, the AI-guided analytics engine sends each user a list of screening questions about the SDoH that they are looking for help with—e.g. access to fresh food, exercise facilities, transportation, housing, income support, etc. The screening questions indicate what aspects of a given SDoH that individual is concerned about and needs to be addressed. Those answers are then sent back to the AI-guided analytics engine which analyses the health/clinical meaning of the answers in the context of the rest of that users data in their POHR 185 and in the context of the relevant peer reviewed literature about that SDoH. Finally, the guidance options are sent back to the user, the user's health coach and PCP for shared decision-making discussions about what steps to take next and how to execute those steps, including how to use the social services entities in the Directory.

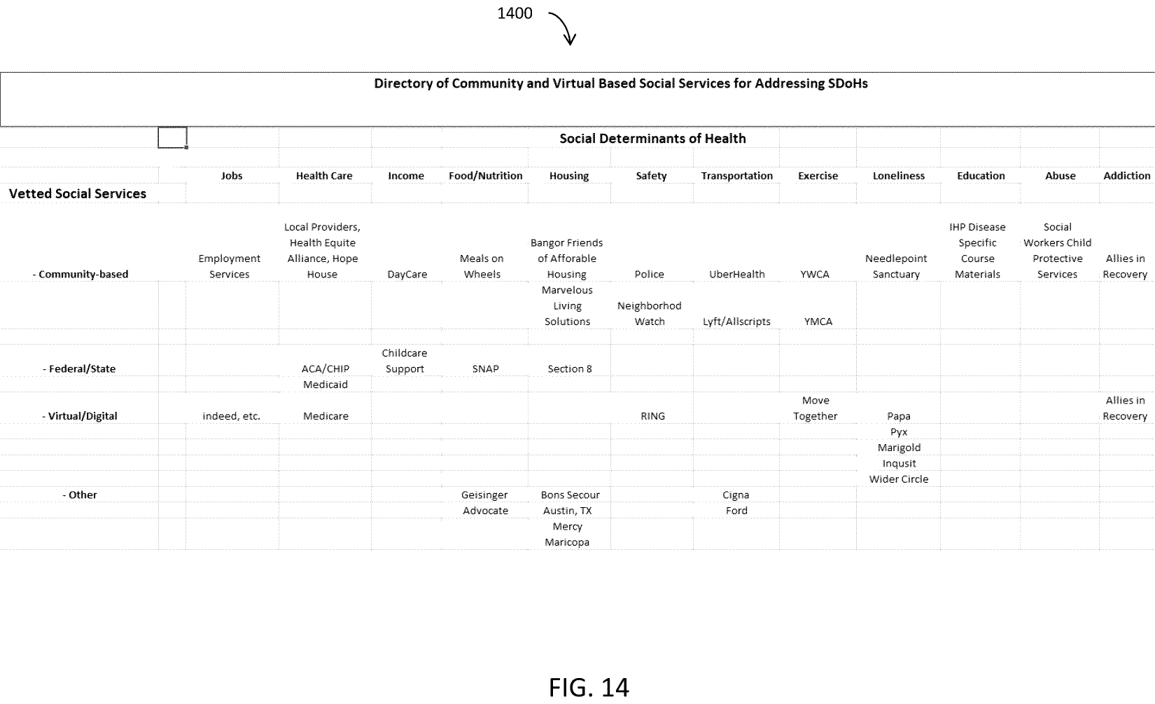

FIG. 14 shows an example of a directory of community and virtual based social services 1400, where vetted social services are shown for an individual's SDoHs.

Linking a patient's SDoHs to Community-based Social Services is a very important aspect of providing health and care guidance because it addresses one of the major problems with current health care delivery, which is that even after collecting data on a health issue, analyzing associated data to create guidance options, and participating in shared decision making to determine which option to pursue, a patient may not perform as agreed because of a lack of access to resources required to perform the agreed upon guidance options. The system overcomes this by creating and providing a Directory of Community-based Social Services as part of each patients POHR 185, which allows the patient, the health coach, or other system user to find resources, for example, transportation, parcel delivery, food delivery, or other resources that might be required to perform certain guidance options.

### Communication System

In addition to being a data, analytics, guidance and documentation platform, the system 100 provides a communication platform for users on a patient's health team to be able to communicate easily with each other while viewing the same data about a particular acute and/or chronic issue a patient may be having. Communication can happen automatically when, for example, a new piece of vetted data is added to the CIHP user's POHR 185—e.g. new medication, lab result, patient terminal 105 device measure, etc. and those given access to the user's CIHP are alerted, if necessary, to the presence of the new data. Communication can also be active whereby one of the approved members of a user's CIHP can message the user with questions, suggestions, requests, etc. Communication may occur among various patient terminals 105, for example, land line phones, mobile phones, tablets, computers, or any suitable electronic device. In some embodiments, communication may occur using paper documents, through fax facilities, mail carriers, or other hardcopy based communication methods.

Communication also occurs between members of the patient's health team when there is a larger amount of data, or written text that requires a larger screen to view the message, such as when guidance options are sent through an external interface 125 of a patient terminal 105, for example, a web based user interface to a laptop computer patient terminal 105.

### Communication Using a Mobile Device

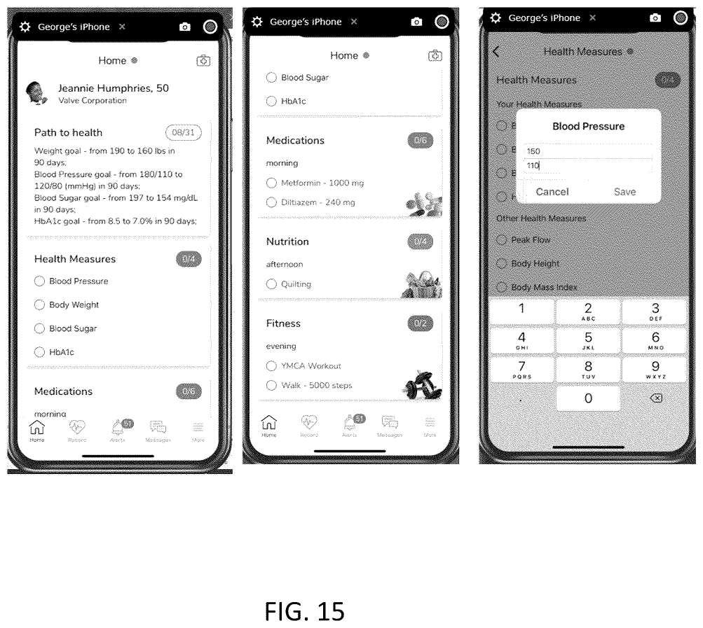

FIGS. 15-21 illustrate examples of system communications using mobile devices. In FIG. 15, after collecting a patient's data and setting health goals & care plans, the patient may view the health goals & care plans and may start taking and recording daily health measures, for example, blood pressure, and may record items related to medications, nutrition and fitness plans.

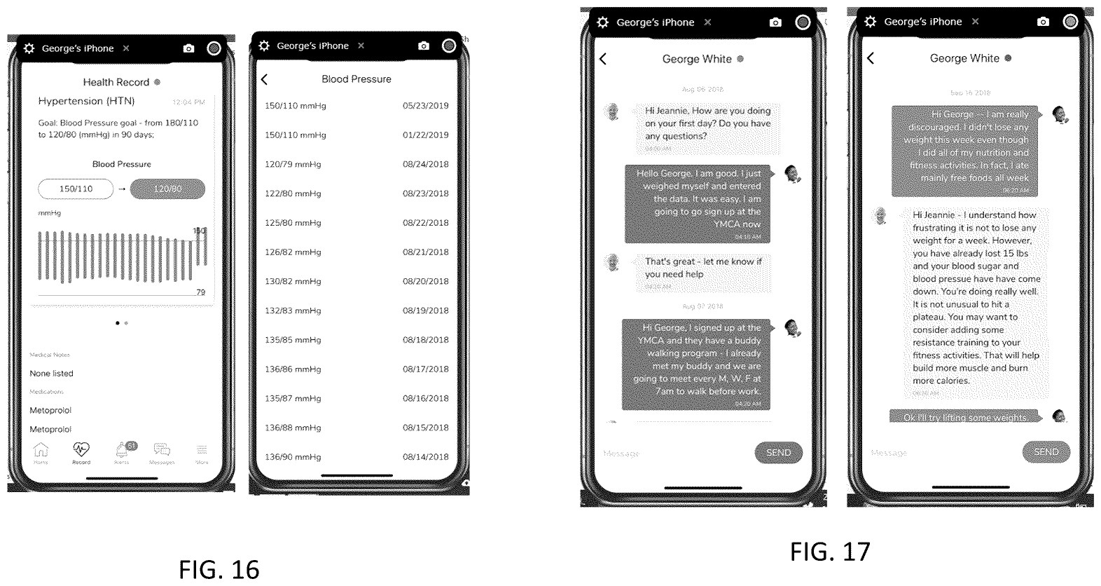

FIG. 16 shows documentation and recording provisions, where each click a user makes on a mobile device may be documented and recorded by the adaptive knowledge engine 1300—e.g. daily health measures, bar charts to show the patient what progress they are making, medication management, nutrition & fitness plan activities, texting with the health coach and other team members, guidance/triage options and accompanying documentation, and peer reviewed journal articles on which the guidance/triage options are based.

FIG. 17 illustrates another communication tool where the patient has the ability to text, email and talk with their health coach and other members of their care team as needed—e.g. the health coach can text the patient to remind them to do something, and the patient can text the health coach to request some help or guidance, etc.

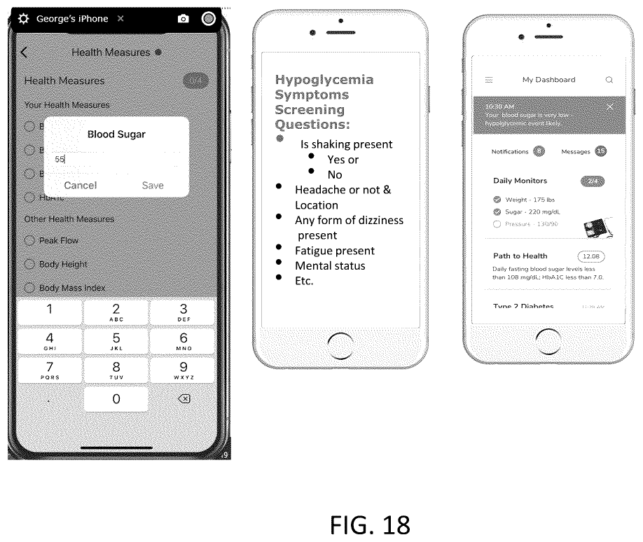

FIG. 18 depicts an example where the system communication capability may provide individualized predictive analysis, where after a low blood glucose measurement of 55, a patient may receive a hypoglycemia screening survey with questions based on literature and the patient's unique health history to assess the patient's risk of becoming hypoglycemic and corrective measures. Where the patient's answers indicate that they are becoming hypoglycemic, the adaptive knowledge engine 1300 may send a triage notification via the patient terminal that the blood glucose level is low and a consultation with the patient's PCP is required. At the same time, the system may generate an alert to the patient's PCP and health coach that the consultation is required. Thus, the system provides earlier detection, better prevention measures, slows disease progression, avoids emergency room or other unplanned medical facility visits, and provides the health coach with the ability to monitor and manage the patient's health.

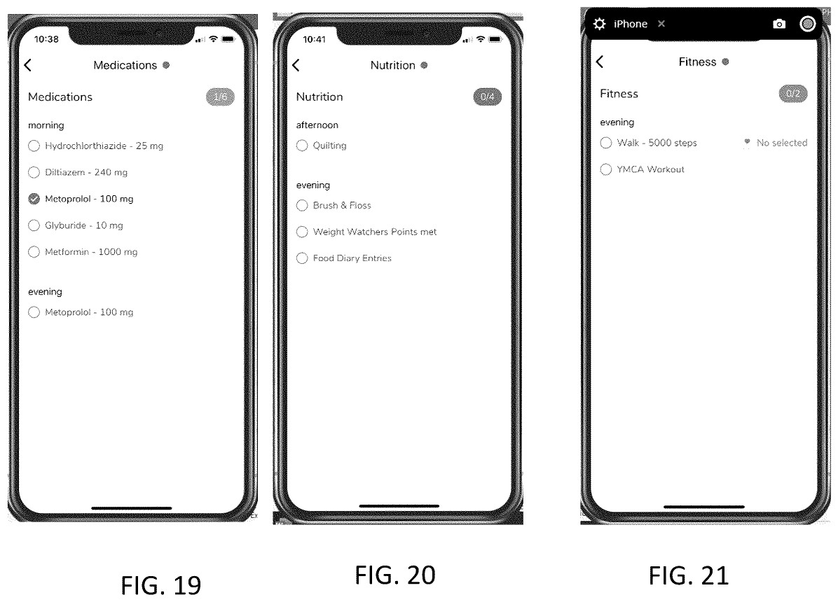

FIG. 19 shows another feature of the system where the adaptive knowledge engine 1300 has found best practice peer reviewed literature that indicates that as a black woman with hypertension, the patient would do better to take a calcium channel blocker, Diltiazem, than a beta blocker, Metoprolol, which the patient is currently taking. This message is sent to the patient, the health coach and PCP to recommend changing the patient's medication program.

As shown in FIG. 20, the patient, working with the patient's data and health coach, has developed a nutrition program to suite the patient's tastes, health needs and schedule, and as part of the program has selected quilting as an activity to relax, and something that is fun and enjoyable and reduces the urge to eat between meals.

FIG. 21 illustrates how, as part of the nutrition program, the patient has selected an aggressive exercise target of 5000 steps per day. The indicator, for example, a red heart, indicates that the patient may import fitness tracking data from another mobile device, for example, a smart watch.

Communication Using a Device with a Larger Display

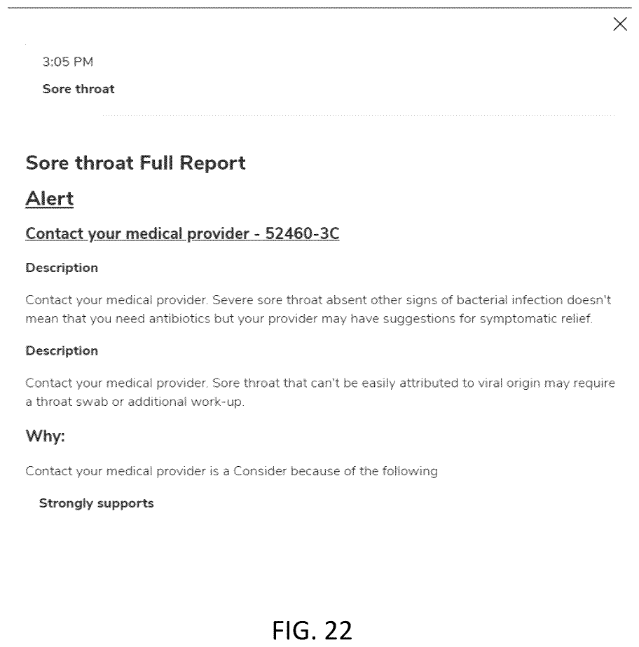

FIGS. 22-24 illustrate examples of system communications using a patient terminal 105 with a larger display, for example, a laptop or personal computer.

As shown in FIG. 22, while the system is sending the guidance/triage options and the short documentation explaining why this guidance/triage option is being sent to the patient, the system may also send a longer version of the documentation to the patient's PCP, health coach's system that use a web based interface, and typically a larger display. This documentation may include relevant differential diagnosis, messaging associated with the diagnosis, an explanation as to why the diagnosis is being considered, relevant guidelines and protocols, peer reviewed literature references, and additional questions generated by the adaptive knowledge engine 1300 to guide the clinician in further assessing both this diagnosis and all others in the queue. The process can be repeated as needed to reach an actionable level of confidence.

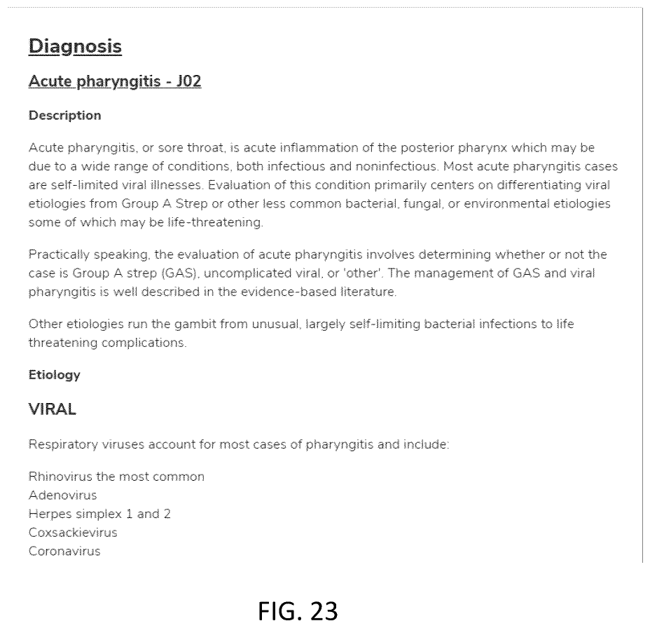

As shown in FIG. 23, this documentation may also include a recall of the data collected, triage reports to aid physician extenders in communicating findings to the clinician, or documentation of encounters at the physician level. If the patient has other active conditions in their problem list, that is included as well. Time of completion may be similar to a traditional interview. Documentation time and completeness is substantially improved.

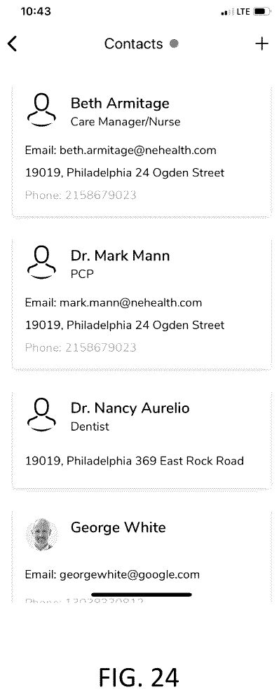

FIG. 24 illustrates that access to contact information for the patient's caregivers may be provided in case they need to be contacted directly.

### Addressing On-Going Health and Care Issues

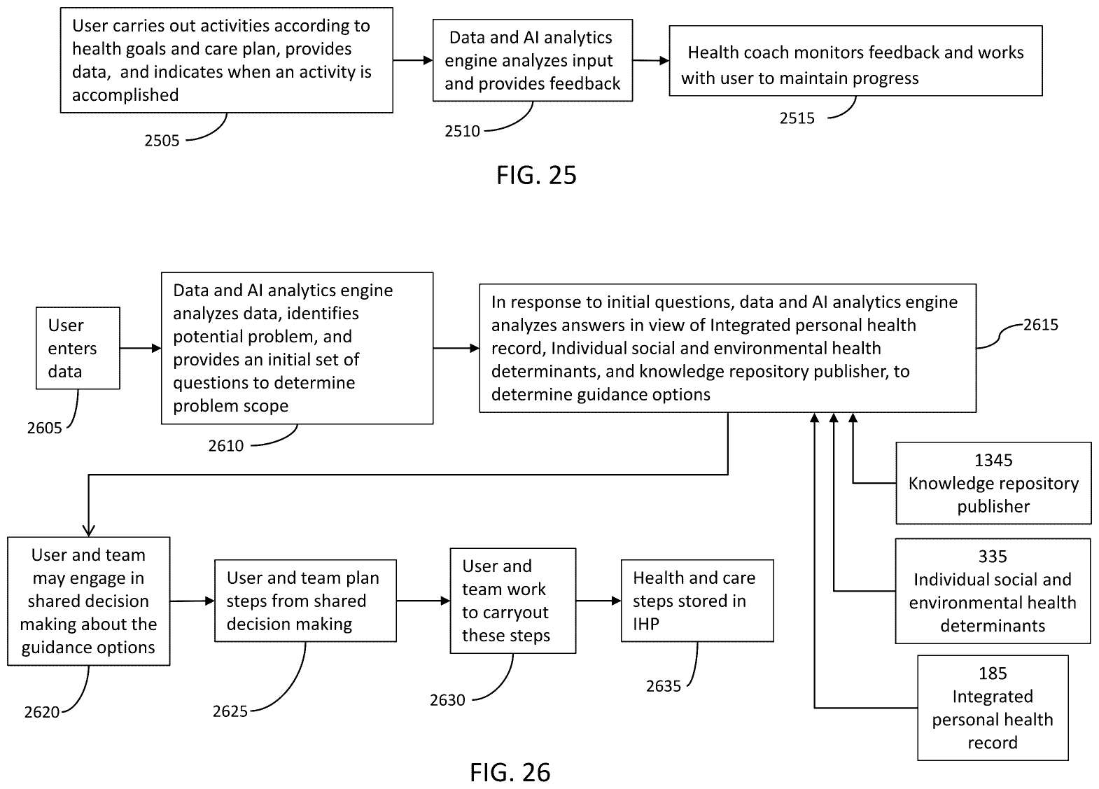

FIG. 25 illustrates an exemplary overview of how, once the health goals and care plan are set, the patient may proceed to carry out their daily health activities—e.g. health measures, medication, nutrition and fitness activities. The patient may use a patient terminal 105 to input readings and other data and to indicate when a particular activity is done, as shown in FIGS. 15-21 described above, and as shown in block 2505. The data and analytics system 160 may analyze the patient's input against the stored health goals and care plan, and may provide feedback, as shown in block 2510. For example, the data and analytics system 160 may send the patient progress reports of how well the patient is doing versus their health goals. The health coach may monitor each activity and may assist the patient in maintaining their goals, as shown in block 2515. For example, the health coach may remind the patient when they have not done a particular activity, may congratulate them on their progress toward achieving their health goals, and may discuss issues the patient is having with carrying out particular health activity.

As shown in FIG. 26, the data and analytics system 160 may also operate to identify a particular health problem from new data entered by a patient. As shown in block 2605, a patient may enter data, for example, while following previously determined health goals, or as a result of identifying a potential health problem. As shown in block 2610, the data and analytics system 160 may analyze the data, may identify a potential problem and may present the patient with a screening survey for the potential health problem. The screening survey may pose refinement and qualifying questions from the medical literature about the possible health problem. As shown in block 2615, the answers may provide an individualized, more precise and thus more accurate description of the patient's condition. The data and analytics system 160 may then perform an analysis in view of the patient's POHR 185, individual social and environmental health determinants 335, and information in the knowledge repository publisher 1345, to determine individualized guidance options. As shown in block 2620, the patient and team may engage in shared decision making about the guidance options, and as shown in block 2625, the patient and team may plan steps resulting from the shared decision making. The patient and team may then work to carry out these steps, as shown in block 2630, and as shown in block 2635, health and care steps may be stored in the patient's CIHP as part of the ongoing documentation, tracking and reporting of what goes on in each CIHP.

As discussed below, all this activity with the health coach, the data, analysis, questions and answers, etc. may be documented by the system's documentation system so that patients, providers and payers can easily see who did what, when, why the guidance provided for a particular micro-health encounter was produced, based on which questions and on which studies.

It should be noted that the POHR 185 for each patient may be continuously updated with longitudinal data files of the individual patient's health and care experience. The resulting knowledge accumulated regarding the individual patient's successful health and care experiences, the bulk data vetted and curated by the adapter translator 1245, and the contents of the knowledge repository 1250 and the lexicon of ontologies 1255 published by the knowledge repository publisher 1265, may be utilized by the AI analytics engine 1305 to generate and update the data analytics warehouse 1315, which in turn may be fed back through the knowledge importer 1240 to the knowledge repository 1250.

The data analytics warehouse 1315 may also be updated from the patient's onboarding POHR shown in block 340 of the on boarding FIG. 5, from the onboarding health and care plan from block 510 of FIG. 5, and data from the patient terminals 105 of FIG. 1.

The updated data analytics warehouse information for each individual patient may be used to further fine tune treatment options for each individual patient. In machine learning terms, every data item stored in the system may be considered every time against every result known to the system in order to ensure maximum fidelity.

The knowledge repository 1250 generally analyzes up-to-date coded and date and time stamped data from peer reviewed literature on subjects related to that issue so that the adaptive knowledge engine 1200 can associate those data elements on those subjects with data used to describe the nature of the acute or chronic issue being considered. The other precondition for the Adaptive knowledge engine 1200 being able to produce refinement questions is that the most up-to date coded and date and time stamped patient data must also be available for the adaptive knowledge engine 1200 to access.

For example, if a patient is complaining of dizziness and shortness of breath (which are coded and time and date stamped), the adaptive knowledge engine 1200 extracts from both the relevant peer reviewed literature and patient data that data that is related to dizziness and shortness of breath and will formulate follow up questions from these associations and send them to the patient's and health coach's smartphone for the patient to answer and send back to the adaptive knowledge engine 1200, which will then formulate guidance options and the peer reviewed literature that was used to produce them from those answers. All of which are sent to the patient, the patient's health coach and PCP for further discussion and shared decision making and implementation.

From those answers, the rest of the patient's data and relevant peer reviewed literature, the adaptive knowledge engine 1200 will then produce guidance options for the patient, their health coach, and others on the patient's health team to carry out and to assess changes to the patient's health goals and care plan.

To produce the guidance options the adaptive knowledge engine 1200 uses the three sets of coded and date and time stamped data:
The descriptions of the symptoms of the acute or chronic problem(s)
The refinement questions or screening survey questions associated with the symptoms or the out of bounds measure from one of the patient terminals 105 and
The patient's health and care data.

Using the various analytical techniques in the adaptive knowledge engine 1200 referenced elsewhere, the adaptive knowledge engine 1200 then produces the guidance options and arrays them by acuity or the severity of the condition(s) that might be causing the symptoms—e.g. if there is an indication that the patient may have a pulmonary embolism which is highly associated with dizziness and shortness of breath and life threatening, then the guidance options will say right away that the patient has to go to the ER right away. That guidance option will be sent to the ER along with the refinement questions/screening questions and the peer reviewed references so that the ER clinicians can see before the patient arrives what is wrong based on what symptoms, answers to questions and questions that were not answered, and what peer reviewed literature. On the other hand, shortness of breath and dizziness are also caused by less serious conditions such as orthostatic hypotension, which would also be listed on the guidance options as a less urgent situation and with suggestions about what to do to verify if this diagnosis is likely or not.

Thus, the individualized health platforms provide a new model of care and health in which N=1 that shows, through the longitudinal experience of each patient, what works best for that patient, as opposed to a population-based model of care. The health and care management system 100 may provide a continuous collaborative care model which implements an individual approach for contact between the individual and the coach—e.g. some patients may want and need to be in communication, for example, through texting, emails, phone calls, as much as once a day or more to keep on track with respect to their care plans and health goals, medications, fitness, nutrition and other health activities. Other patients may require less frequent contact. The continuous collaborative distributed care model may encourage enhanced participation by individuals in their care and health because it reflects more accurately the way health and care needs express themselves in real life—e.g. differently for each individual and sometimes at unexpected moments-rather than the way care is currently provided through set episodes with a clinician in a bricks and mortar clinic. Early detection, prevention and better management of chronic diseases may require more continuous attention and pursuit of health goals and care plans than the current episodic care models permit. The continuous collaborative care model may begin with the collection of each patient's health and care data from which the patient, the health coach 165, the data and analytics system 160 and the patient's PCP put together the patient's health goals and care plans—e.g. if the patient's data shows that the patient has high blood pressure, one of the patient's health goals will be to lower their too high blood pressure, and the care plan will make recommendations as to the best way to go about achieving that specific health goal—e.g. perhaps through a combination of daily monitoring of blood pressure with a home blood pressure monitor, better nutrition, more fitness activities and a medication program. The health coach 165 and the data and analytics system 160 as informed by the best peer reviewed literature will provide guidance options about possible nutrition, fitness and medication plans for specific health goals and related care plans. In working with the patient and that patient's CIHP 190 to implement the continuous collaborative care model, the health coach 165 may use data guidance options and motivational techniques that each health coach 165 has been taught in their certification program to help the patient find the best way to implement this model and thus achieve their health goals by adhering to their related care plan.

A micro-care encounter may be defined as a unit of measure or analysis that may be performed by a patient, the health coach 165, a patient's physician, or the data and analytics system 160—e.g. it could be the patient taking their daily blood glucose level and entering that measure into their patient terminal 105; or it could be the health coach 165 seeing that value and texting the patient to say that compared to the last six days this latest blood glucose level is headed in the wrong direction; or it could be the data and analytics system 160 sending a patient who has diabetes a hyperglycemia screening survey questionnaire; or it could be the patient clicking on abdominal pain from the list of 25 acute symptom complexes to indicate that there is a problem and receiving back a list of refinement questions on abdominal pain from the literature. There are literally thousands of possible micro-care encounters. The important point here is that the patient can respond right away to an issue that a micro-care encounter flags up—e.g. your latest blood pressure reading is headed in the wrong direction. Because there are in effect, three new providers, the patient, the health coach 165, and the CIHP 190, the use of traditional healthcare services may be reduced by the activities of these three new providers and the incremental cost of subsequent micro-care encounters may be significantly and progressively reduced.

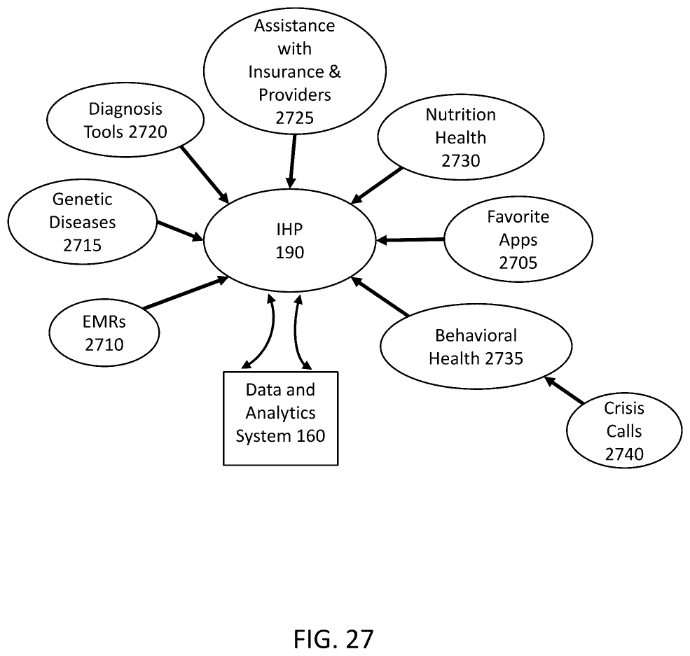

FIG. 27 illustrates that each patient can connect external information and databases to their CIHP 190 that may be used in interactions between the patient's CIHP and the data and analytics system 160, for example through the external interfaces 125₁-125ₙ (FIG. 1). The external information and databases may include—e.g. favorite apps 2705, data from electronic medical records (EMRs) 2710, data from databases about genetic diseases 2715 and other disease states that are not covered in the base set of disease states in each patient's CIHP 190, information and databases related to diagnostic tools 2720, information and databases related to assistance with insurance and providers 2725, information and databases related to nutrition health 2730, information and databases related to behavioral health 2735 and related crisis calls 2740, etc. As noted, every patient is unique with different genomes, physiology, personality, preferences, diseases, medications, etc. The granular, flexible data architecture underlying each CIHP means that each patient can customize what data and capabilities are available to them based on their unique needs—e.g. someone may have behavioral health issues in which case their CIHP and health coach would be put in touch with a vetted Behavioral Health set of capabilities. Similarly, as changes keep occurring in health and care, such as the use of personal genome, metabolome and other-omic data, the flexible data architecture allows the patient's CIHP to, in near real time, adapt its structure and architecture to accommodate this new data, unlike current EMRs which are brittle and more difficult to change. Because of the underlying data and analytics system data architecture, the platform application 175 can connect to the patient's home health monitors, their electronic medical records, and other apps that the patient may like—e.g. favorite recipe or jogging apps for the nutrition and/or fitness part of their health program. In this way, the health and care management system 100 becomes a super-integrator or platform of platforms, vetting and curating each new source of data before the data can enter a given patient's CIHP 190, thus dealing with the garbage in garbage out problem and the explosion of digital health devices of unknown quality, safety and security.]

Each time the patient enters new data into a patient terminal 105, that data may be sent to the data and analytics system 160, which may read and analyze the data and create a new set of guidance options for the patient based on that new data. The guidance options may be displayed in an interactive format with various buttons that may provide additional functionality. for example, if the patient clicks on a refine button provided under a piece of guidance, the data and analytics system 160 may produce a set of refinement questions from the literature for the patient to answer. If the patient clicks on one of any number of acute symptoms, the data and analytics system 160 may produce a set of questions for the patient to answer. As mentioned above, periodically or with the presentation of new troubling data, the data and analytics system 160 may send to the patient's CIHP 190 a set of screening questions about an aspect of one of the patient's health goals or care plan in order to identify early, and to prevent the emergence of, new or chronic health issue e.g. early identification and prevention of hypoglycemia, depression, and many others.

Each new acute/chronic care/health issue and the data on that issue may be assessed and analyzed in the context of all of a given individual's health and care data and in the context of the relevant peer reviewed literature. There are several analytical techniques that the data and analytics system 160 uses to carry out this analysis. The important point here is that the data and analytics system 160 does not rely on one modeling or analytical technique as most other systems do—e.g. many systems use treatment algorithms that are static and based on probability, not optimization of matching to an individual's particular circumstances. This data and analytics system 160 doesn't just rely on algorithms. It uses the best technique for the analytical question to be answered. So, since triage, diagnosis, treatment and management suggestions are going to change with the underlying science, the setting and the individual in question, so too should the analytical approaches change.

Referring again to FIG. 27, the system may include a set of quality and safety guard rails for new data coming into the system from an individual's favorite app to make sure that the new data makes sense and is of high quality—e.g. if a body temperature reading says 200 degrees Fahrenheit it will not be accepted. The vetting of data may be done as the new candidate data comes into the data and analytics system 160. The quality and safety guard rails may include range limits for each measured physiological characteristic of the patient, —e.g. the body temperature example above. The range limits may be derived from the peer reviewed literature and/or protocols that the medical community has established for ranges of specific clinical/heath issues. For example, the range limits may be derived by the adapter translator 1245.

One of the other problems that the present embodiments address is that currently within and between clinics, about 40% of the time patients are referred to the wrong resource to deal with their problem—e.g. patients end up in the offices of cardiovascular doctors when they really should have been sent to a vascular specialist. The CIHPs address this referral problem by doing a better job of diagnosing the cause of the problem for which the patient is being referred in the first place. This better diagnosis occurs because the adaptive knowledge engine 1300 arrives at a differential diagnosis and eventually a diagnosis by assessing the current acute/chronic problem in the context of all the rest of the patient's data in their POHR 185 and in the context of the relevant peer reviewed literature and in the context of the patient's answers to peer reviewed literature produced refinement question or screening surveys to produce an individualized and thus more accurate set of diagnostic options from which more accurate referral suggestions are made if need be.

### External Referral System

Another referral problem occurs when a patient needs a resource or an answer to a question to carry out the next steps suggested by the guidance options and by the shared decision-making interaction between the patient, the health coach, the PCP and others on the patient's new health team. The Directory of Community-based Social Services 1400 can address many of these questions having to do with a patient's SDOH—e.g. access to fresh food, transportation, exercise facilities, social contacts, etc., but there are many types of questions that lie outside of the ability of the Directory's ability to answer such as health care insurance questions, mental health, substance abuse issues, etc. For these types of questions the system has a referral system that vets possible resources that can address the question and then puts the patient or health coach in touch with that resource. The health coach is the person on the patient's new health team that provides these vetted referral services—e.g. putting the patient in touch with someone that is vetted who is an expert in psychiatry for a patient's mental health problem; or someone who is an expert in health insurance options, etc. . . . A vetting process has been developed for selecting high value referral resources that involves doing research on a range of possible resources for a given question, and then winnowing down the list by talking with past patients of a potential resource, using online objective rating services, such as Health Grades,

### Value-Based Payment System

The health and care management system 100 may also implement a value-based payment (VBP) system that financially rewards improved health outcomes. For example, when the patient properly uses their data collection device 130 and achieves improved health outcomes, such as lower blood pressure, blood sugar, more exercise, better oxygen levels, etc., the medical practitioner may be reimbursed a certain amount per patient per month, thus aligning the provider's financial interests with the patient's health outcomes and interests, a situation that only occurs when all concerned are using a value-based payment system as enabled by the use of the CIHPs.

In one embodiment, rather than getting paid for the number or volume of each diagnosis, test, lab, image, treatment, etc. for a given patient, the VBP system may pay medical practitioners for a bundle of services for an episode of care—e.g. instead of just paying for a knee operation, the medical practitioner may be reimbursed for a bundle of services before, during and after the operation, such as pre-op services, the operation itself, and post op rehabilitation, but may only be paid if the activities in the bundle result in a successful outcome or result for the patient—e.g. was the knee bundle of services a success based on success criteria for the knee episode of care bundle of services developed by professional specialist bodies, for example, the American Academy of Orthopedics. In other words, to get paid in VBP systems providers must take on risk that they will be able to perform a bundle of episode of care services at a certain quality level and below a certain cost level.

In these VBP systems if providers make a mistake, they will not get reimbursed for the work they did producing that mistake, which currently in fee-for-service systems they do get paid for. So, the key to the success of VBP systems is being able to improve patients' care and health outcomes, which leads to a reduction in the use of traditional health care services and their associated costs through reductions in preventable medical mistakes, better early detection and prevention and management of chronic diseases. If providers can do this, they then get a portion of the shared savings that result from this chain of events.

IHPs facilitate this VBP process by providing patients and providers with:
A new division of labor wherein the patient, their health coach and their CIHP become new care providers and members of the patient's new health team producing much of the data vetting, recall, refinement and analysis, and guidance option and documentation preparation that clinicians who are ill suited for these tasks have traditionally done, thus reducing clinician burden considerably,
The data, tools and systems they need to improve upstream quality of data, analysis and guidance options that lead to better more timely decisions, fewer preventable medical errors, better early detection and prevention and management of chronic diseases, all of which leads to a reduction in the use of traditional population-based health care services and a rise in less expensive virtual, digital, individualized health care services and an improvement in care, health and cost outcomes,
The cost savings, a portion of which is used to reimburse providers and patients for their degree of success in improving care, health, and cost outcomes one patient at a time, and
The data on quality, cost, and value of each acute bundle of care and/or each chronic disease bundle of care needed to calculate cost savings and share of these savings going back to providers and patients.

The various data components of each micro-care encounter may facilitate a transition to a value-based payment system. Over time, by being able to provide the systems, and health coaches and the resulting granular data on costs, quality by micro-cost encounter, the CIHPs will enable patients to do a better job of early detection, prevention, and management of chronic/acute health issues. CIHPs will also enable providers to have the data and tools they need to improve their care and their patients' health outcomes—the combination of which will result in a reduction in the use of traditional healthcare services and their associated costs, thus entitling physicians to receive a share of the cost savings from this kind of VBP system. In this way, medical practitioners' financial interests and patient health interests are aligned—e.g. medical practitioners do better financially, if their patients become healthier.

As discussed below, all this activity with the health coach, the data, analysis, questions and answers, etc. may be documented in each patient's POHR 185 so that patients, providers and payers can easily see who did what, when, why the guidance provided for a particular micro-health encounter was produced, based on which questions and on which studies.

Through this model, the individual and the health coach learn from their experience with each other and from the results of the data review performed by the data and analytics system 160, which determinants of health may be more important to a given individual. For example, while population level protocols and guidelines change frequently for health indicators such as cholesterol, blood pressure, lipid levels, etc., often the important thing for the individual is what level of a particular health indicator works best for them in terms of their own health situation, health and care goals and plans—e.g. it may be that for some people a blood pressure goal of 140—instead of the 120 population guideline—is more appropriate. These insights also help to drive more robust engagement by the individual in their care and health. While there is much talk about standardizing care, this is inimical to the reality that each of us has a different body with different health and care needs.

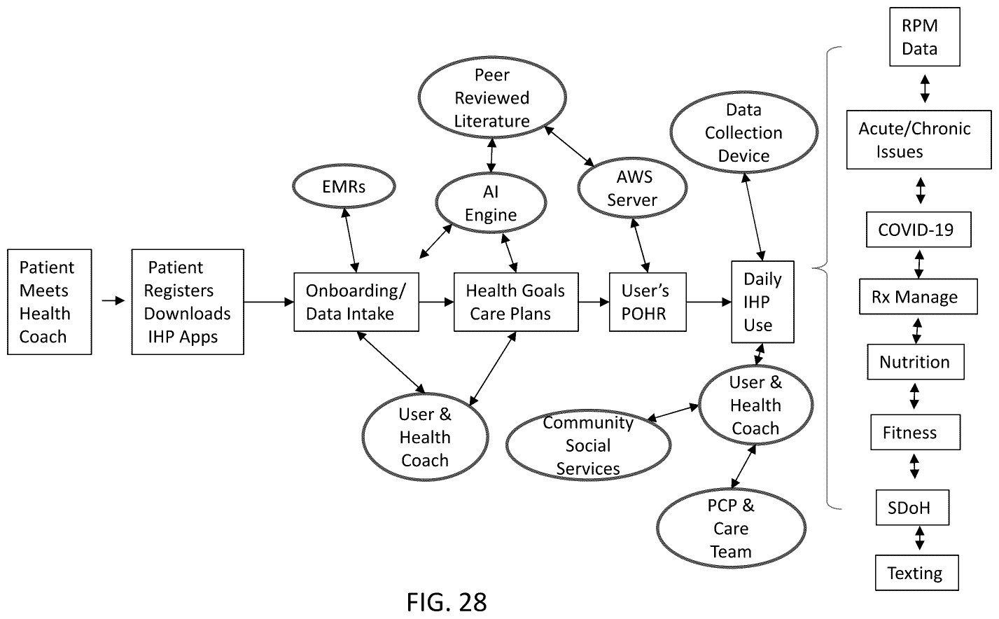

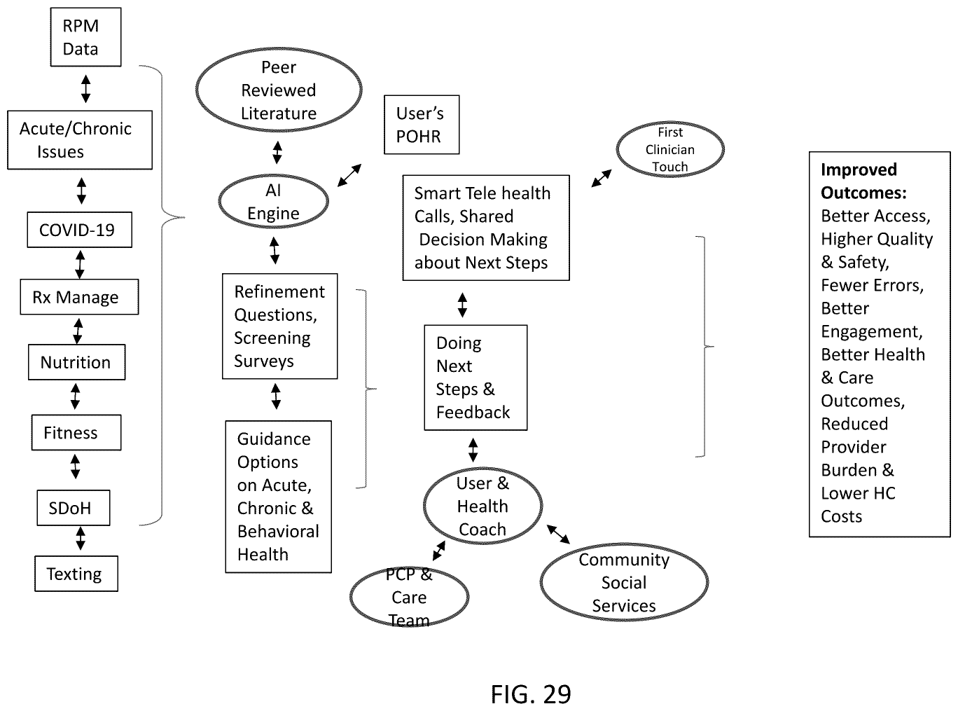

FIG. 28 illustrates a summary of the set up steps, various assets, and particular health and care issues that may be managed for a specific patient through their individualized CIHP.

FIG. 29 illustrates a summary of how the system operates on an ongoing basis to address changes to the particular health care issues where the data and analytics system 160 may analyze the data, may identify a potential problem, may present the patient with a screening survey, and provide updated guidance options for the potential health problem.

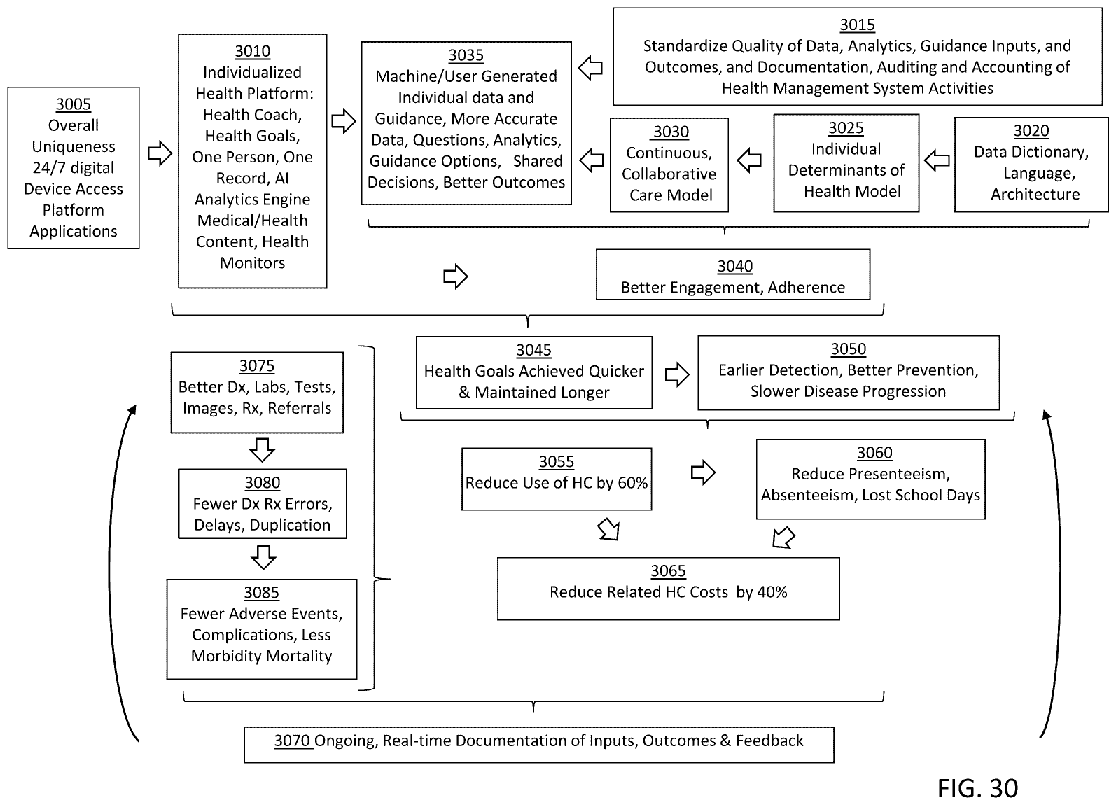

FIG. 30 illustrates at least some of the advantages provided by the disclosed health and care management system 100:

Overall Uniqueness, Access, and Platform Applications: (Block 3005) There are thousands of single shot digital mobile health monitors, wearables and sensors on the market today, but the CIHPs 190 are the only 24/7 patient terminal device accessible, evidence-based, AI-guided, comprehensive, portable, digital health platforms that bring together and integrate all the resources, including home health monitors, that an individual needs to manage and maximize their own individual health and care, and thus improve their own access, quality, safety, engagement, health, care and cost outcomes. In this way, the CIHPs 190 enable each person to become both a provider and consumer of their own health and care efforts—or the new “prosumers” of health and care—chaperoned by the quality and safety guardrails imbedded in the data vetting and contextualized, individual guidance options produced by the data and analytics system 160. Flowing from the nature of the human body, health and care themselves, CIHPs are the only integrated, comprehensive health and care platform that enables the drivers and determinants of each person's health to interact with each other in an interdependent manner mirroring the interdependent way the organ systems and other aspects of each person's body interact with each other. CIHPs also recognize that each person's particular circumstances of life, family, work, health etc. are unique, and so the tools in each person's CIHP need to be taken out to that individual to provide them with the agency they need to find their own path to improved health and care. Every other approach basically asks the individual to come to the clinic, join this or that wellness/disease management program. CIHPs take exactly the opposite approach and give the individual the tools they need to fashion their own approach. Each individual may have access to and ownership of their record of their entire health and care history to the extent possible—e.g. one person one record, unlike the current practice of individuals having their data scattered across a number of different electronic medical records (electronic medical records) with a number of different providers.

The system 100 includes platform applications 175₁-175ₙ that enable patients to access their CIHP 190 and a health coach 165 at all times for many different health and care related activities. While most of the day-to-day health and care tasks that a patient will want to carry out may be accomplished on relatively smaller devices, such as smartphones, there are some tasks for which larger devices are better suited because of their larger screen sizes. These activities may include the initial collection of the patient's health and care data, viewing the full report and background data, references and documentation underlying specific guidance options. The platform applications 175₁-175ₙ may operate to adapt the size and amount of information displayed based on the display technology of the patient terminal 105, for example, displaying less data with limited data manipulation and reporting on a smaller device while displaying more data with more data manipulation and reporting capabilities on larger devices. Because of the underlying data and analytics system data architecture, the platform application 175 can connect to the patient's home health monitors, their electronic medical records, and other apps that the patient may like—e.g. favorite recipe or jogging apps for the nutrition and/or fitness part of their health program. In this way, the health management system 100 becomes a super-integrator or platform of platforms, vetting and curating each new source of data before the data can enter a given patient's CIHP 190, thus dealing with the garbage in garbage out problem and the explosion of digital health devices of unknown quality, safety and security.

Community-based Individualized Health Platform: (Block 3010) As described in detail above, the—health management system 100 provides a community-based individualized health platform for each patient that may include among other things, a health coach, health goals, a one person, one record problem-oriented health record model where N=1, an AI analytics engine and health monitors. The CIHPs 190 are unique because they are also the only health platforms that mimic the individual, holistic interdependent nature of the human body and thus human health. Each of us has a unique genome, physiology, psychology, living, family and work situation and thus a unique set of health, care, preference and scheduling needs.

### Problem Oriented Health Record

For decades, health care delivery has been plagued first by paper records and then by electronic records both of which recorded what happened to a patient in a siloed fashion—e.g. the data was organized by type of activity such as by labs, tests, images, treatments, referrals, etc. or by provider. In both cases, the data does not give a concise, comprehensive picture of what problems a patient has had/is having and what was done for each problem. The POHR 185 operates to organize all a patient's data from all vendors and providers in one record by problem and then within each problem what happened to the patient for that problem chronologically. In this way, the physician, the patient, or anyone else on the patient's care team can quickly see what problems the patient has had/is still dealing with, how they were treated, are there any gaps in care. By bringing all a patient's data together into one record and then organizing the data by problem, POHRs 185 are a significant step toward improving our healthcare delivery system.

Standardize the Quality of the Data, Analytics and Guidance Inputs and Outcomes, and the Documentation, Auditing and Accounting of the Health Management System Activities: (Block 3015) The healthcare industry is the only major industry that does not have standards for the quality of the data, analytics and guidance inputs and outcomes, and for the documentation, auditing and accounting of input, outcomes and processes. The health management system 100 has created de facto standards for the industry in lieu of the federal and/or state governments creating these standards, and thus is the only system that has these much-needed standards. Standards for the quality of the data, analytics and guidance inputs and outcomes, and for the documentation, auditing and accounting of system activities: establish de facto standards for the quality of the data, analytics, guidance inputs and outcomes, and for the documentation, auditing and accounting of the health management system 100 activities in taking care of an individual's health and care micro-encounters and needs. As a result, the quality of the initial vetted input data for each health and care issue, and the ongoing collected data is enhanced.

Health/Care Data Dictionary, Language, Ontology and Architecture: (Block 3020) The data and analytics system 160 provides a case entity personal health record, an analytics engine and health/medical content development parts of the CIHPs 190 and has as its foundation a very flexible, modular, granular health/medical data dictionary, language, ontology and architecture, that makes it possible to establish bi-directional data exchanges between the CIHPs 190 and other sources of health/care data such as the external data collection devices 130, electronic medical records, health monitors, etc. provided that the data coming from the sending source of data has standardized, coded data. The data and analytics system 160 vets and curates the data coming from these other data feeds before allowing them to become part of an individual's CIHP 190, thus providing the quality and safety guardrails missing from current systems.

Utilization of Individual Determinants: (Block 3025) As explained above, the system may operate to collect a list of individual social and environmental health determinants for use in developing the patient's health and care plan.

Continuous/Collaborative, Distributed Care Model, Better Engagement and Management of Chronic Diseases: (Block 3030) With 24/7 patient terminal device access to their CIHPs 190 and the continuous support of their health coaches, patients are able to put together realistic health goals and care plans, identify emerging issues earlier, prevent the onset of new chronic diseases, manage their existing chronic diseases more effectively and thus slow down the progression of their chronic diseases from one stage to another. The continuous nature of CIHPs also sets them apart because they are able to much more effectively help the patient and the health coach set health goals and care plans, work on achieving them on a daily basis by following through on the activities that the guidance options say should be carried out. Currently, with existing systems about 50% of a patient's instructions are ignored and never carried out.

Machine Generated Individual Data and Guidance, More Accurate Data, Questions, Analytics, Guidance Options, Shared Decisions, Better Outcomes: (Block 3035) The CIHPs 190 are the only systems that have the multiple computerized data collection, data vetting, data refinement and data analytics capabilities to assess each new acute, chronic and/or prevention health/care issue in the context of the rest of that individual's health/care data and in the context of the relevant peer reviewed health/medical literature. No one, including clinicians, can “remember” all this data, carry out this type of multi-factorial analysis and produce individual (not sub-population protocols) guidance options that are free of human cognitive biases, such as confirmation, anchoring and/or many other cognitive biases, which cause many of the medical errors that harm millions of people each year.

By being the only systems that individualize the data inputs and the resulting guidance options, the CIHPs 190 are the only systems that, therefore, produce much more precise and accurate data and the foundation for shared decision making between CIHP patients, their health coaches, primary care physicians and other clinicians on their care team. These better decisions in turn result in far fewer mistakes, adverse events, complications, trips to the ER, hospitals and other clinical settings. All of which may result in a reduction in the use of traditional healthcare services and a drop in associated healthcare costs.

Better Engagement and Adherence—Take Health/Care Resources Out to Each Individual: (Block 3040) The CIHPs 190 are the only approach that takes this integrated set of health/care resources, out to each person in their own particular circumstances of living, family and work to enable each person with their health coach to figure out what is the best way for them to use these resources and become more involved in their care and health.

Health Goals Achieved More Quickly and Maintained Longer: (Block 3045) The system facilitates the patient becoming more involved, invested and motivated to achieve their health goals.

Earlier Detection, Better Prevention, Slower Disease Progression: (Block 3050) Each patient's POHR 185 may be implemented as an integrated health and care data base that may include all an individual's medical history integrated in one record, that promotes prevention, earlier problem detection, and better disease management that results in slower disease progression.

Better Care, Health and Cost Outcomes: as a result, individual's use of traditional healthcare services may be reduced by approximately 60%, (Block 3055), presenteeism and absenteeism may be reduced (Block 3060), and related healthcare costs may be reduced by approximately 40%, (Block 3065).

Ongoing Documentation, Feedback and Continuous Process Improvement: (Block 3070) each CIHP 190 tracks, documents, audits and accounts for each click that the individual and/or coach does—e.g. who did what, based on which data, when, why, with what outcomes. This documentation is sent on a continuous basis back up into the AI-driven health, clinical analytics engine for constant learning and process improvement.

Additional system advantages include Better Diagnoses (Dx), Lab Results, Tests, Images, Treatments (Rx), Referrals, (Block 3075), Fewer Diagnosis and Treatment Errors, Delays, Duplication, (Block 3080), and Fewer Adverse Events, Complications, Less Morbidity Mortality, (Block 3085).

The above described components of the health management system 100 work together so that for a given care encounter or health issue, these systems produce the following improvements over current best practices:

Security of Patient's Health/Care Data: Beyond the encrypted data security protocols that the AWS servers use and that the various components may utilize, the data and analytics system 160 operates to separate a patient's personal identifier data from their health/care data. As a result, even if someone were able to hack into a patient's account they would not be able to put the personal identifier data together with their health/care data.

Holistic System for Contextualized Health/Care: The human body is also a highly complex system of interdependent biological systems, which means that every new acute, chronic and/or prevention health/care issue that occurs for a given individual needs to be assessed, as the CIHPs 190 do, in the context of that individual's entire health/care history, their existing chronic conditions, active acute issues, current medications, vital signs, allergies and other aspects of that person's health/care.

Force Multiplier, Task Shifting, New Division of Labor, Reduce Shortage of Clinicians: The CIHPs 190 thus create three new providers, the patient, the patient's system (CIHP) and the patient's health coach. Together, these providers, operating the health and care management system 100, can relieve clinicians of all the above data collection, data vetting, data refinement, data analytics and guidance option preparation tasks. These providers may also use the system 100 to facilitate documentation of what happens on each platform and during each patient encounter—e.g. each click, what happened, with what results, with what inputs, who did it, why, based on which references, etc. In this way, these providers represent a new health/care force multiplier at a time when millions of people worldwide are having trouble accessing high quality, safe care and health assistance because of shortages of nurses, primary care physicians and other clinicians.

Relief for Clinicians, Lessons Learned, Continuous Process Improvement and Learning: The above clerical tasks of data collection, etc. are tasks for which clinicians, relative to these three new providers, are ill-suited to perform. We have asked clinicians to do these tasks that are humanly impossible and/or turn highly educated individuals into documentation clerks, which is one of the reasons why clinicians are so dissatisfied with their current situation with resulting high levels of substance abuse, mental illness and suicide. With this new task shifting and division of labor, clinicians are now freed up to focus on more complicated patients and on analyzing the outcomes data from the documentation produced by the CIHPs 190 for best practices, lessons learned which are fed back up into the AI-analytics engine for continuous process improvement and learning. This continuous provision of best practice data may also reduce legal medical liability and insurance claims.

Higher Quality Refinement of that Data: Better refinement questions on that data, because the questions come from the vetted health/clinical literature not clinicians, who are human and thus have cognitive biases and limited memory capabilities,

Higher Quality Analysis of that Data: Better analysis by the AI-driven health/clinical analytics engine of the health/care meaning of the new data in the context of all the other data in that person's health database and in the context of the relevant health/medical literature,

Precision Health and Care Guidance Options and Shared Decision Making: More individual, accurate and therefore precise (i.e. precision health and medicine) guidance options and possible next steps for the individual patient to discuss with their health coach, PCP and other members of their care team for shared decision making,

Easier Access and Better Engagement in Health and Care: Because of easier 24/7 access through their smartphone and other digital devices to their CIHP 190, the setting of realistic care plans and health goals with their health coach, more ongoing continuous contact with their health coach through sometimes daily nudges, more shared decision making about their care and health issues, individuals become more involved in their care and health, which in turn generates the following three streams of powerful improvements in care, health and cost outcomes:

Better health and care decisions about nutrition, fitness, chronic disease management, diagnoses, labs, images, tests, treatments, acute issues.

Fewer errors, adverse events, complications and trips to the ER, PCP or specialists.

Better adherence to care plans and quicker and more sustained achievement of health goals.

Better individual predictive analytics-earlier detection of emerging care and health issues, better prevention of the onset of new issues, and slowing down of progression of existing chronic diseases.

The new division of labor and task shifting produced by the use of the CIHPs 190 will produce significant benefits for clinicians or health providers:
They will no longer be required to remember all the patient's data and related health and medical literature and try to link the two sets of data to determine guidance options—an impossible task for anyone;
As a result, of not having to perform these tasks, for which they are ill suited relative to the CIHPs 190, they will be able to focus their time and attention on tasks for which they are better suited, e.g. spending more time on shared decision making, spending more time on complicated patients, spending more time on analyzing the documentation results for process learning and process improvement; and
Because the system provides continuous best practice rationale for guidance options, clinicians medical liability risks and insurance premiums will be reduced.

COVID-19 Screening, Analysis, Diagnosis, and Risk Stratification module within context of the rest of the patient's data and in the context of the relevant evidence-based content on multiple respiratory diseases and on other possible causes of symptoms. The adaptive knowledge engine 1300 uses this data to assess patients' answers to screening questions to determine whether a patient is at risk for COVID-19 or for something else and if so what risk level are they at and what steps should they take next. This capability can also be used for other pandemics.

Various modifications and adaptations may become apparent to those skilled in the relevant arts in view of the foregoing description, when read in conjunction with the accompanying drawings. However, all such and similar modifications of the teachings of the disclosed embodiments will still fall within the scope of the disclosed embodiments.

Various features of the different embodiments described herein are interchangeable, one with the other. The various described features, as well as any known equivalents can be mixed and matched to construct additional embodiments and techniques in accordance with the principles of this disclosure.

Furthermore, some of the features of the exemplary embodiments could be used to advantage without the corresponding use of other features. As such, the foregoing description should be considered as merely illustrative of the principles of the disclosed embodiments and not in limitation thereof.

What is claimed is:

1. A system for providing a patient with an individualized health platform, comprising:
a data and analytics system including:
one or more patient problem-oriented health records including a data structure comprising:
a top level problem view organized by health and care issues including a patient's current and past chronic and acute problems;
a detail level problem view for each chronic and acute problem organized by each step in a diagnostic, treatment and management process;
a top level status view of the health and care issues;
a detail level status view of each health and care issue; and
a patient's social and environmental health determinants;
an adaptive knowledge engine configured to automatically import health and clinical data for use in analyzing the one or more patient problem-oriented health records and assessing diagnostic, treatment, and management options for the health and care issues;
a patient terminal for providing the patient with an ability to interact with the system;
one or more patient data collection devices coupled to the patient terminal; and
a directory of community and virtual-based social services comprising vetted social service organizations for use by the patient in addressing one or more of the patient's social and environmental health determinants.

2. The system of claim 1, wherein the top level problem view is further organized by bodily systems.

3. The system of claim 1, wherein the detail level problem view is organized in order of each step in a diagnostic and treatment and management process as a series of micro-care encounters arranged chronologically.

4. The system of claim 1, wherein the top level status view is further organized by active and inactive status by bodily systems.

5. The system of claim 1, wherein the detail level status view is organized by a status of a particular health and care issue.

6. The system of claim 1, wherein the social and environmental health determinants comprise main factors and driving factors for each main factor, the main factors comprising:
healthcare factors, individual behavior factors, socio-economic factors, personal security issues, individual physiology factors, and environmental factors.

7. The social and environmental health determinants of claim 6, wherein:
the driving factors for the healthcare factors comprise improved early detection, prevention, and management of primary care for acute care and chronic diseases, end-of-life care, health information technology, and health care financing and incentives;
the driving factors for the individual behavior factors comprise character, intelligence, substance abuse, sleep patterns, nutrition, exercise, stress, social contacts, and social support; and
the driving factors for socio-economic factors comprise family situations; income, employment, neighborhood, housing, education, and services.

8. The social and environmental health determinants of claim 6, wherein:
the driving factors for the personal security factors comprise the condition of the patient's neighborhood and their housing, crime level, police protection, security, neighborhood watch organizations;
the driving factors for the individual physiology factors comprise a patient's genome, metabolome, physical and mental disabilities, and chronic illnesses; and
the driving factors for the environmental factors comprise the patient's neighborhood air and water quality, presence of toxic waste, and positive culture of the neighborhood.

9. The system of claim 1, wherein the adaptive knowledge engine is configured to automatically import health and clinical data for use in analyzing the patient problem oriented health records and assessing diagnostic, treatment and management options, the adaptive knowledge engine comprising:
a source document repository for storing peer reviewed health and clinical data;
an artificial intelligence analytics engine configured to operate on the peer reviewed health and clinical data to provide updated diagnostic, treatment, and management options for the health and care issues.

10. The system of claim 9, wherein the adaptive knowledge engine further comprises an adapter translator configured to perform validity checks on the imported health and clinical data.

11. The system of claim 1, wherein the one or more data collection devices comprise a medication dispenser comprising circuitry that signals the adaptive knowledge engine when a medication has been dispensed.

12. A method of providing a patient with an individualized health platform, comprising:
receiving agreement to terms and conditions for use from the patient;
assigning a health coach to the patient;
receiving permission to access the patient's medical practitioner records from the patient;
collecting the patient's previously undocumented health and care data;
collecting the patient's individual social and environmental health determinants;
using a data and analytics system to organize the collected health and care data into a problem oriented health record for the patient comprising records relating to a particular health and care issue arranged as a series of micro care encounters comprising units of measure or analysis relating to the particular health and care issue and organized in order of each step in a diagnostic and treatment and management process in chronological order; and
using an adaptive knowledge engine to automatically import health and clinical data for use in analyzing the problem-oriented health record and assessing diagnostic, treatment, and management options for the health and care issues.

13. The method of claim 12, further comprising:
having the patient work with the health coach to draft health goals and a care plan including activities to address the health and care issue;
using the data and analytics system to vet the health goals and care plan by comparing the health goals and care plan against peer-reviewed health and medical literature recommended actions;
providing the individualized health goals and care plan vetted and curated by the data and analytics system to the patient's primary care physician for vetting and approval;
storing the vetted and approved individualized health goals and care plan in the patient's problem oriented health record.

14. The method of claim 13, further comprising:
having the patient input an indication that a particular activity to address the health and care issue is complete; and
using the data and analytics system to analyze the indication against the vetted and approved individualized health goals and care plan, and provide feedback to the patient regarding progress toward the health goals.

15. The method of claim 13, further comprising:
using the data and analytics system to:
analyze additional data input by the patient;
identify an additional health problem from the additional data;
present a screening survey to the patient requesting additional information related to the additional health problem;
analyze the additional information in view of the patient's problem oriented health record, individual social and environmental health determinants, and the peer-reviewed health and medical literature recommended actions; and
determine guidance for addressing the additional health problem.
